(19) United States
(12) Patent Application Publication
(10) Pub. No.: US 2023/0340598 A1
(43) Pub. Date: Oct. 26, 2023

(54) Association Of T Cell Leukemia/Lymphoma Protein 1A (TCL1A) With Clonal Hematopoiesis Of Indeterminate Potential (CHIP)

(71) Applicant: Regeneron Pharmaceuticals, Inc., Tarrytown, NY (US)

(72) Inventors: Eric Jorgenson, Tarrytown, NY (US); Michael Kessler, Tarrytown, NY (US); Amy Damask, Tarrytown, NY (US); Sean O’Keeffe, Tarrytown, NY (US)

(21) Appl. No.: 18/051,488

(22) Filed: Oct. 31, 2022

Related U.S. Application Data
(60) Provisional application No. 63/274,388, filed on Nov. 1, 2021.

(51) Int. Cl. C12Q 1/6883

(52) U.S. Cl. CPC C12Q 1/6883; C12Q 1/6869

(57) ABSTRACT

Methods of treating subjects having clonal hematopoiesis of indeterminate potential (CHIP) with T Cell Leukemia/Lymphoma Protein 1A (TCL1A) antagonists, and methods of identifying subjects having an increased or decreased risk of developing CHIP are provided herein.

### REFERENCE TO SEQUENCE LISTING

This application includes a Sequence Listing filed electronically as an XML file named 381203632SEQ, created on Oct. 27, 2022, with a size of 25 kilobytes. The Sequence Listing is incorporated herein by reference.

### FIELD

The present disclosure relates generally to the treatment of subjects having clonal hematopoiesis of indeterminate potential (CHIP) due to somatic mutations in, for example, Tet Methylcytosine Dioxygenase 2 (TET2), and/or ASXL Transcriptional Regulator 1 (ASXL1), with TCL1A antagonists, and methods of determining the risk of developing CHIP in subjects having TCL1A variants.

### BACKGROUND

CHIP is a genetically defined phenotype reflecting age-related changes to hematopoietic stem cells (HSCs). As a person ages, their HSCs accumulate mutations as a result of DNA replication error and DNA damage repair (so called somatic mutations, such as those acquired after birth). Thus, prevalence rises with age and is roughly 10% among persons aged 70 to 80. Patients undergoing molecular genetic investigation for cytopenia (anemia, leukopenia, thrombocytopenia) are the most likely to be given this diagnosis. Some of these mutations confer growth advantages, which result in: increased proliferation of these cells relative to other cells, increase in frequency of these mutations, and accumulation of additional mutations that drive neoplastic changes. A subset of genes are strongly recurrently mutated along with clonal hematopoiesis; these are considered “CHIP genes” and they include: DNA Methyltransferase 3 Alpha (DNMT3A), Tet Methylcytosine Dioxygenase 2 (TET2), ASXL Transcriptional Regulator 1 (ASXL1), Janus kinase 2 (JAK2), and Splicing factor 3B subunit 1 (SF3B1). CpG => TpG mutations are very common in CHIP. In addition to the identification in blood DNA of specific recurrent mutations, the clinical definition of CHIP requires the absence of dysplasia and leukemia (< 20% blasts). CHIP is associated with increased risk of hematologic cancers, such as myeloid or lymphoid neoplasia, and with increased risk of atherosclerotic cardiovascular disease, such as coronary heart disease, myocardial infarction, and severe calcified aortic valve stenosis.

TCL1A is an oncogene whose product, TCL1, is a 13 kDa protein whose function requires it to form homodimers. TCL1 acts as co-activator of AKT kinases and mediates normal growth and survival signals when physiologically expressed. When TCL1A is dysregulated, it causes lymphomagenesis and cancer progression. TCL1 is a prominent isoform of the TCL1 family proteins that are involved in the normal development of early Band T-cells. The expression of TCL1 has been described in germinal center (GC) centroblast, centrocyte and post-GC memory B cells, in tumors arising from the germinal center such as follicular lymphoma (FL), Burkitt lymphoma (BL), diffuse large B cell lymphoma (DLBCL), and from memory cells such as chronic lymphocytic leukemia (CLL). Prolonged and increased expression of TCL1 in the late phases of thymocyte development causes T cell prolymphocytic leukemia (T-PLL). TCL1 dysregulation in T cells is due to a chromosomal translocation that brings TCL1 (on chromosome 14q31.2) under TCR (T Cell Receptor) enhancer elements. The precise mechanisms underlying the over-expression of TCL1 in B cell tumors are unclear.

### SUMMARY

The present disclosure provides methods of treating, preventing, or reducing the development of CHIP in a subject, the methods comprising administering a TCL1A antagonist to the subject.

The present disclosure also provides methods of treating a subject with a therapeutic agent that treats, prevents, or reduces development of CHIP, wherein the subject has CHIP or is at risk of developing CHIP, and wherein the subject comprises a TET2-CHIP mutation, the methods comprising: determining whether the subject has a TCL1A variant nucleic acid molecule by: obtaining or having obtained a biological sample from the subject; and performing or having performed a sequence analysis on the biological sample to determine if the subject has a genotype comprising TCL1A variant nucleic acid molecule; and administering or continuing to administer the therapeutic agent that treats, prevents, or reduces development of CHIP in a standard dosage amount to a subject that is TCL1A reference; and/or administering a TCL1A antagonist to the subject; administering or continuing to administer the therapeutic agent that treats, prevents, or reduces development of CHIP in an amount that is the same as, greater than, or less than a standard dosage amount to a subject that is heterozygous for the TCL1A variant nucleic acid molecule; and/or administering a TCL1A antagonist to the subject; or administering or continuing to administer the therapeutic agent that treats, prevents, or reduces development of CHIP in an amount that is the same as, greater than, or less than a standard dosage amount to a subject that is homozygous for the TCL1A variant nucleic acid molecule; wherein the presence of a genotype having the TCL1A variant nucleic acid molecule indicates the subject has a decreased risk of developing CHIP.

The present disclosure also provides methods of treating a subject with a therapeutic agent that treats, prevents, or reduces development of CHIP, wherein the subject has CHIP or is at risk of developing CHIP, and wherein the subject comprises an ASXL1-CHIP mutation, the methods comprising: determining whether the subject has a TCL1A variant nucleic acid molecule by: obtaining or having obtained a biological sample from the subject; and performing or having performed a sequence analysis on the biological sample to determine if the subject has a genotype comprising TCL1A variant nucleic acid molecule; and administering or continuing to administer the therapeutic agent that treats, prevents, or reduces development of CHIP in a standard dosage amount to a subject that is TCL1A reference; and/or administering a TCL1A antagonist to the subject; administering or continuing to administer the therapeutic agent that treats, prevents, or reduces development of CHIP in an amount that is the same as, greater than, or less than a standard dosage amount to a subject that is heterozygous for the TCL1A variant nucleic acid molecule; and/or administering a TCL1A antagonist to the subject; or administering or continuing to administer the therapeutic agent that treats, prevents, or reduces development of CHIP in an amount that is the same as, greater than, or less than a standard dosage amount to a subject that is homozygous for the TCL1A variant nucleic acid molecule; wherein the presence of a genotype having the TCL1A variant nucleic acid molecule indicates the subject has a decreased risk of developing CHIP.

The present disclosure also provides methods of identifying a subject having an increased risk of developing CHIP, wherein the subject comprises a DNMT3A-CHIP mutation, the methods comprising: determining or having determined the presence or absence of a TCL1A variant nucleic acid molecule in a biological sample obtained from the subject; wherein: when the subject is TCL1A reference, then the subject has a decreased risk of developing CHIP compared to a subject that comprises the TCL1A variant nucleic acid molecule; and when the subject is heterozygous or homozygous for the TCL1A variant nucleic acid molecule, then the subject has an increased risk of developing CHIP compared to a subject that is TCL1A reference.

The present disclosure also provides methods of identifying a subject having a decreased risk of developing CHIP, wherein the subject comprises a TET2-CHIP mutation, the methods comprising: determining or having determined the presence or absence of a TCL1A variant nucleic acid molecule in a biological sample obtained from the subject; wherein: when the subject is TCL1A reference, then the subject has an increased risk of developing CHIP compared to a subject that comprises the TCL1A variant nucleic acid molecule; and when the subject is heterozygous or homozygous for the TCL1A variant nucleic acid molecule, then the subject has a decreased risk of developing CHIP compared to a subject that is TCL1A reference.

The present disclosure also provides methods of identifying a subject having a decreased risk of developing CHIP, wherein the subject comprises an ASXL1-CHIP mutation, the methods comprising: determining or having determined the presence or absence of a TCL1A variant nucleic acid molecule in a biological sample obtained from the subject; wherein: when the subject is TCL1A reference, then the subject has an increased risk of developing CHIP compared to a subject that comprises the TCL1A variant nucleic acid molecule; and when the subject is heterozygous or homozygous for the TCL1A variant nucleic acid molecule, then the subject has a decreased risk of developing CHIP compared to a subject that is TCL1A reference.

The present disclosure also provides methods of identifying a subject having an increased risk of developing a solid tumor, the methods comprising: determining or having determined the presence or absence of at least one CHIP somatic mutation at a high variant allele fraction (VAF) in a biological sample obtained from the subject; wherein: when the subject has a high VAF of at least one CHIP somatic mutation, then the subject has an increased risk of developing the solid tumor; and when the subject does not have a high VAF of at least one CHIP somatic mutation, then the subject does not have an increased risk of developing the solid tumor.

The present disclosure also provides methods of identifying a subject having an increased risk of developing a blood cancer, the methods comprising: determining or having determined the presence or absence of at least one CHIP somatic mutation at a high VAF in a biological sample obtained from the subject; wherein: when the subject has a high VAF of at least one CHIP somatic mutation, then the subject has an increased risk of developing the blood cancer; and when the subject does not have a high VAF of at least one CHIP somatic mutation, then the subject does not have an increased risk of developing the blood cancer.

### BRIEF DESCRIPTION OF THE DRAWINGS

The accompanying figures, which are incorporated in and constitute a part of this specification, illustrate several features of the present disclosure.

FIG. 1 shows forest plots featuring results from survival analyses in UKB. Panel A shows that TET2 mutation carriers were at the greatest risk of developing blood cancers (HR = 4.70 [3.86-5.72], P = 1.50 * 10-53), whereas DNMT3A mutation carriers had much more modest risk of acquiring blood cancers (HR = 1.70 [1.39-2.07], P = 3.00 * 10-7) unless they also had at least one additional CHIP mutation (HR = 3.28 [2.29-4.69], P = 9.90 * 10-11). When decomposing blood cancers into myeloid and lymphoid subtypes, it was estimated that high VAF CHIP carriers were at a significantly elevated risk of developing myeloid cancers (HR = 6.92 [6.10-7.86], P = 1.20 * 10⁻¹⁹⁵, Panel B) compared with lymphoid cancers (HR = 1.57 [1.26-1.94], P = 3.90 * 10⁻⁵, Panel C). Panel D shows data for breast cancer. Panel E shows data for colon cancer. Panel F shows data for lung cancer. Panel G shows data for prostate cancer. Panel H shows that DNMT3A carriers are only at a significantly increased risk of blood cancer in non-smokers. Panel I shows associations driven by DNMT3A and ASXL1 CHIP carriers, with both estimated to have elevated lung cancer risk in both smokers and non-smokers. Panel J shows that the risk of death from any cause is significantly elevated among high VAF CHIP carriers (1.29 [1.21-1.38], P = 2.10 × 10⁻¹⁵), and is similar across DNMT3A, TET2, and ASXL1 CHIP subtypes.

FIG. 2 shows gene expression across hematopoietic cells for TCL1A.

LENGTHY TABLES
The patent contains a lengthy table section. A copy of the table is available in electronic form from the USPTO web site ( https://seqdata.uspto.gov/?pageRequest=docDetail&DocID=US20230340598A1 ). An electronic copy of the table will also be available from the USPTO upon request and payment of the fee set forth in 37 CFR 1.19(b)(3).

### DESCRIPTION

Various terms relating to aspects of the present disclosure are used throughout the specification and claims. Such terms are to be given their ordinary meaning in the art, unless otherwise indicated. Other specifically defined terms are to be construed in a manner consistent with the definitions provided herein.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-expressed basis for interpretation, including matters of logic with respect to arrangement of steps or operational flow, plain meaning derived from grammatical organization or punctuation, or the number or type of aspects described in the specification.

As used herein, the singular forms “a,” “an” and “the” include plural referents unless the context clearly dictates otherwise.

As used herein, the term “about” means that the recited numerical value is approximate and small variations would not significantly affect the practice of the disclosed embodiments. Where a numerical value is used, unless indicated otherwise by the context, the term “about” means the numerical value can vary by ±10% and remain within the scope of the disclosed embodiments.

As used herein, the term “comprising” may be replaced with “consisting” or “consisting essentially of” in particular embodiments as desired.

As used herein, the term “isolated”, in regard to a nucleic acid molecule or a polypeptide, means that the nucleic acid molecule or polypeptide is in a condition other than its native environment, such as apart from blood and/or animal tissue. In some embodiments, an isolated nucleic acid molecule or polypeptide is substantially free of other nucleic acid molecules or other polypeptides, particularly other nucleic acid molecules or polypeptides of animal origin. In some embodiments, the nucleic acid molecule or polypeptide can be in a highly purified form, i.e., greater than 95% pure or greater than 99% pure. When used in this context, the term “isolated” does not exclude the presence of the same nucleic acid molecule or polypeptide in alternative physical forms, such as dimers or Alternately phosphorylated or derivatized forms.

As used herein, the terms “nucleic acid”, “nucleic acid molecule”, “nucleic acid sequence”, “polynucleotide”, or “oligonucleotide” can comprise a polymeric form of nucleotides of any length, can comprise DNA and/or RNA, and can be single-stranded, double-stranded, or multiple stranded. One strand of a nucleic acid also refers to its complement.

As used herein, the term “subject” includes any animal, including mammals. Mammals include, but are not limited to, farm animals (such as, for example, horse, cow, pig), companion animals (such as, for example, dog, cat), laboratory animals (such as, for example, mouse, rat, rabbits), and non-human primates. In some embodiments, the subject is a human. In some embodiments, the human is a patient under the care of a physician.

It has been observed in accordance with the present disclosure that the presence in a subject (having a DNMT3A-CHIP mutation) of a genotype having a TCL1A variant nucleic acid molecule indicates the subject has an increased risk of developing CHIP. It has also been observed in accordance with the present disclosure that the presence in a subject (having a TET2-CHIP mutation) of a genotype having a TCL1A variant nucleic acid molecule indicates the subject has a decreased risk of developing CHIP. It has also been observed in accordance with the present disclosure that the presence in a subject (having an ASXL1-CHIP mutation) of a genotype having a TCL1A variant nucleic acid molecule indicates the subject has a decreased risk of developing CHIP.

It has also been observed in accordance with the present disclosure that a subject that has a high variant allele fraction (VAF) of at least one CHIP somatic mutation has an increased risk of developing a solid tumor. It has also been observed in accordance with the present disclosure that a subject that has a high VAF of at least one CHIP somatic mutation, then the subject has an increased risk of developing a blood cancer.

The present disclosure provides methods of treating, preventing, or reducing the development of CHIP in a subject, the methods comprising administering a TCL1A antagonist to the subject.

The present disclosure also provides methods of treating a subject with a therapeutic agent that treats, prevents, or reduces development of CHIP, wherein the subject has CHIP or is at risk of developing CHIP, and wherein the subject comprises a DNMT3A-CHIP mutation. These methods comprise: determining whether the subject has a TCL1A variant nucleic acid molecule by: obtaining or having obtained a biological sample from the subject, and performing or having performed a sequence analysis on the biological sample to determine if the subject has a genotype comprising TCL1A variant nucleic acid molecule. In some embodiments, these methods further comprise administering or continuing to administer the therapeutic agent that treats, prevents, or reduces development of CHIP in a standard dosage amount to a subject that is TCL1A reference, and/or administering a TCL1A antagonist to the subject. In some embodiments, these methods further comprise administering or continuing to administer the therapeutic agent that treats, prevents, or reduces development of CHIP in an amount that is the same as, less than, or greater than a standard dosage amount to a subject that is heterozygous for the TCL1A variant nucleic acid molecule, and/or administering a TCL1A antagonist to the subject. In some embodiments, these methods further comprise administering or continuing to administer the therapeutic agent that treats, prevents, or reduces development of CHIP in an amount that is the same as, less than, or greater a standard dosage amount to a subject that is homozygous for the TCL1A variant nucleic acid molecule. The presence of a genotype having the TCL1A variant nucleic acid molecule indicates the subject has an increased risk of developing CHIP.

The present disclosure also provides methods of treating a subject with a therapeutic agent that treats, prevents, or reduces development of CHIP, wherein the subject has CHIP or is at risk of developing CHIP, and wherein the subject comprises a TET2-CHIP mutation. These methods comprise: determining whether the subject has a TCL1A variant nucleic acid molecule by: obtaining or having obtained a biological sample from the subject, and performing or having performed a sequence analysis on the biological sample to determine if the subject has a genotype comprising TCL1A variant nucleic acid molecule. In some embodiments, these methods further comprise administering or continuing to administer the therapeutic agent that treats, prevents, or reduces development of CHIP in a standard dosage amount to a subject that is TCL1A reference, and/or administering a TCL1A antagonist to the subject. In some embodiments, these methods further comprise administering or continuing to administer the therapeutic agent that treats, prevents, or reduces development of CHIP in an amount that is the same as, less than, or greater than a standard dosage amount to a subject that is heterozygous for the TCL1A variant nucleic acid molecule, and/or administering a TCL1A antagonist to the subject. In some embodiments, these methods further comprise administering or continuing to administer the therapeutic agent that treats, prevents, or reduces development of CHIP in an amount that is the same as, less than, or greater a standard dosage amount to a subject that is homozygous for the TCL1A variant nucleic acid molecule. The presence of a genotype having the TCL1A variant nucleic acid molecule indicates the subject has a decreased risk of developing CHIP.

The present disclosure also provides methods of treating a subject with a therapeutic agent that treats, prevents, or reduces development of CHIP, wherein the subject has CHIP or is at risk of developing CHIP, and wherein the subject comprises a ASXL1-CHIP mutation. These methods comprise: determining whether the subject has a TCL1A variant nucleic acid molecule by: obtaining or having obtained a biological sample from the subject, and performing or having performed a sequence analysis on the biological sample to determine if the subject has a genotype comprising TCL1A variant nucleic acid molecule. In some embodiments, these methods further comprise administering or continuing to administer the therapeutic agent that treats, prevents, or reduces development of CHIP in a standard dosage amount to a subject that is TCL1A reference, and/or administering a TCL1A antagonist to the subject. In some embodiments, these methods further comprise administering or continuing to administer the therapeutic agent that treats, prevents, or reduces development of CHIP in an amount that is the same as, less than, or greater than a standard dosage amount to a subject that is heterozygous for the TCL1A variant nucleic acid molecule, and/or administering a TCL1A antagonist to the subject. In some embodiments, these methods further comprise administering or continuing to administer the therapeutic agent that treats, prevents, or reduces development of CHIP in an amount that is the same as, less than, or greater a standard dosage amount to a subject that is homozygous for the TCL1A variant nucleic acid molecule. The presence of a genotype having the TCL1A variant nucleic acid molecule indicates the subject has a decreased risk of developing CHIP.

In any of the embodiments described herein, the subject has or is at risk of developing a hematologic cancer, a myeloid neoplasia, a lymphoid neoplasia, an atherosclerotic cardiovascular disease, a coronary heart disease, a myocardial infarction, or severe calcified aortic valve stenosis. In any of the embodiments described herein, the CHIP or CHIP-related disorder is a hematologic cancer, a myeloid neoplasia, a lymphoid neoplasia, an atherosclerotic cardiovascular disease, a coronary heart disease, a myocardial infarction, and/or a severe calcified aortic valve stenosis. In any of the embodiments described herein, the CHIP or CHIP-related disorder is a hematologic cancer, a myeloid neoplasia, or a lymphoid neoplasia. In some embodiments, the CHIP or CHIP-related disorder is a hematologic cancer. In some embodiments, the CHIP or CHIP-related disorder is a myeloid neoplasia. In some embodiments, the CHIP or CHIP-related disorder is a lymphoid neoplasia. In some embodiments, the CHIP or CHIP-related disorder is an atherosclerotic cardiovascular disease. In some embodiments, the CHIP or CHIP-related disorder is a coronary heart disease. In some embodiments, the CHIP or CHIP-related disorder is a myocardial infarction. In some embodiments, the CHIP or CHIP-related disorder is a severe calcified aortic valve stenosis.

In some embodiments, the subject has a DNMT3A-CHIP somatic mutation. In any of the embodiments described herein, the DNMT3A-CHIP somatic mutation can include variations at positions of chromosome 2 using the nucleotide sequence of the DNMT3A reference genomic nucleic acid molecule (ENSG00000119772.17, chr2:25,227,855-25,342,590 in the GRCh38/hg38 human genome assembly) as a reference sequence. Exemplary variants is provided below.

In some embodiments, the subject has an ASXL1-CHIP somatic mutation. In any of the embodiments described herein, the ASXL1-CHIP somatic mutation can include variations at positions of chromosome 20 using the nucleotide sequence of the ASXL1 reference genomic nucleic acid molecule (ENSG00000171456.20, chr20:32,358,330-32,439,260 in the GRCh38/hg38 human genome assembly) as a reference sequence.

In some embodiments, the subject has a TET2-CHIP somatic mutation. In any of the embodiments described herein, the TET2-CHIP somatic mutation can include variations at positions of chromosome 4 using the nucleotide sequence of the TET2 reference genomic nucleic acid molecule (ENSG00000168769.14, chr4:105,146,293-105,279,816 in the GRCh38/hg38 human genome assembly) as a reference sequence.

In some embodiments, the subject is administered a TCL1A antagonist. In some embodiments, the TCL1A antagonist comprises an inhibitory nucleic acid molecule that hybridizes to a TCL1A nucleic acid molecule. Examples of inhibitory nucleic acid molecules include, but are not limited to, antisense nucleic acid molecules, small interfering RNAs (siRNAs), and short hairpin RNAs (shRNAs). Such inhibitory nucleic acid molecules can be designed to target any region of a TCL1A nucleic acid molecule. In some embodiments, the antisense RNA, siRNA, or shRNA hybridizes to a sequence within a TCL1A genomic nucleic acid molecule or mRNA molecule and decreases expression of the TCL1A polypeptide in a cell in the subject. In some embodiments, the TCL1A antagonist comprises an antisense molecule that hybridizes to a TCL1A genomic nucleic acid molecule or mRNA molecule and decreases expression of the TCL1A polypeptide in a cell in the subject. In some embodiments, the TCL1A antagonist comprises an siRNA that hybridizes to a TCL1A genomic nucleic acid molecule or mRNA molecule and decreases expression of the TCL1A polypeptide in a cell in the subject. In some embodiments, the TCL1A antagonist comprises an shRNA that hybridizes to a TCL1A genomic nucleic acid molecule or mRNA molecule and decreases expression of the TCL1A polypeptide in a cell in the subject.

The inhibitory nucleic acid molecules can comprise RNA, DNA, or both RNA and DNA. The inhibitory nucleic acid molecules can also be linked or fused to a heterologous nucleic acid sequence, such as in a vector, or a heterologous label. For example, the inhibitory nucleic acid molecules can be within a vector or as an exogenous donor sequence comprising the inhibitory nucleic acid molecule and a heterologous nucleic acid sequence. The inhibitory nucleic acid molecules can also be linked or fused to a heterologous label. The label can be directly detectable (such as, for example, fluorophore) or indirectly detectable (such as, for example, hapten, enzyme, or fluorophore quencher). Such labels can be detectable by spectroscopic, photochemical, biochemical, immunochemical, or chemical means. Such labels include, for example, radiolabels, pigments, dyes, chromogens, spin labels, and fluorescent labels. The label can also be, for example, a chemiluminescent substance; a metal-containing substance; or an enzyme, where there occurs an enzyme-dependent secondary generation of signal. The term “label” can also refer to a “tag” or hapten that can bind selectively to a conjugated molecule such that the conjugated molecule, when added subsequently along with a substrate, is used to generate a detectable signal. For example, biotin can be used as a tag along with an avidin or streptavidin conjugate of horseradish peroxidate (HRP) to bind to the tag, and examined using a calorimetric substrate (such as, for example, tetramethylbenzidine (TMB)) or a fluorogenic substrate to detect the presence of HRP. Exemplary labels that can be used as tags to facilitate purification include, but are not limited to, myc, HA, FLAG or 3XFLAG, 6XHis or polyhistidine, glutathione-S-transferase (GST), maltose binding protein, an epitope tag, or the Fc portion of immunoglobulin. Numerous labels include, for example, particles, fluorophores, haptens, enzymes and their calorimetric, fluorogenic and chemiluminescent substrates and other labels.

The inhibitory nucleic acid molecules can comprise, for example, nucleotides or non-natural or modified nucleotides, such as nucleotide analogs or nucleotide substitutes. Such nucleotides include a nucleotide that contains a modified base, sugar, or phosphate group, or that incorporates a non-natural moiety in its structure. Examples of non-natural nucleotides include, but are not limited to, dideoxynucleotides, biotinylated, aminated, deaminated, alkylated, benzylated, and fluorophor-labeled nucleotides.

The inhibitory nucleic acid molecules can also comprise one or more nucleotide analogs or substitutions. A nucleotide analog is a nucleotide which contains a modification to either the base, sugar, or phosphate moieties. Modifications to the base moiety include, but are not limited to, natural and synthetic modifications of A, C, G, and T/U, as well as different purine or pyrimidine bases such as, for example, pseudouridine, uracil-5-yl, hypoxanthin-9-yl (I), and 2-aminoadenin-9-yl. Modified bases include, but are not limited to, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo (such as, for example, 5-bromo), 5-trifluoromethyl and other 5-substituted uracils and cytosines, 7-methylguanine, 7-methyladenine, 8-azaguanine, 8-azaadenine, 7-deazaguanine, 7-deazaadenine, 3-deazaguanine, and 3-deazaadenine.

Nucleotide analogs can also include modifications of the sugar moiety. Modifications to the sugar moiety include, but are not limited to, natural modifications of the ribose and deoxy ribose as well as synthetic modifications. Sugar modifications include, but are not limited to, the following modifications at the 2′ position: OH; F; O—, S—, or N-alkyl; O—, S—, or N-alkenyl; O—, S— or N-alkynyl; or O-alkyl-O-alkyl, wherein the alkyl, alkenyl, and alkynyl may be substituted or unsubstituted C₁₋₁₀alkyl or C₂₋₁₀alkenyl, and C₂₋₁₀alkynyl. Exemplary 2′ sugar modifications also include, but are not limited to, —O[(CH₂)ₙO]ₘCH₃, —O(CH₂)ₙOCH₃, —O(CH₂)ₙNH₂, —O(CH₂)ₙCH₃, —O(CH₂)ₙ—ONH₂, and —O(CH₂)ₙON[(CH₂)ₙCH₃)]₂, where n and m, independently, are from 1 to about 10. Other modifications at the 2′ position include, but are not limited to, C₁₋₁₀alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars can also include those that contain modifications at the bridging ring oxygen, such as CH₂ and S. Nucleotide sugar analogs can also have sugar mimetics, such as cyclobutyl moieties in place of the pentofuranosyl sugar.

Nucleotide analogs can also be modified at the phosphate moiety. Modified phosphate moieties include, but are not limited to, those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. These phosphate or modified phosphate linkage between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts, and free acid forms are also included. Nucleotide substitutes also include peptide nucleic acids (PNAs).

In some embodiments, the antisense nucleic acid molecules are gapmers, whereby the first one to seven nucleotides at the 5′ and 3′ ends each have 2′-methoxyethyl (2′-MOE) modifications. In some embodiments, the first five nucleotides at the 5′ and 3′ ends each have 2′-MOE modifications. In some embodiments, the first one to seven nucleotides at the 5′ and 3′ ends are RNA nucleotides. In some embodiments, the first five nucleotides at the 5′ and 3′ ends are RNA nucleotides. In some embodiments, each of the backbone linkages between the nucleotides is a phosphorothioate linkage.

In some embodiments, the siRNA molecules have termini modifications. In some embodiments, the 5′ end of the antisense strand is phosphorylated. In some embodiments, 5′-phosphate analogs that cannot be hydrolyzed, such as 5′-(E)-vinyl-phosphonate are used. In some embodiments, the siRNA molecules have backbone modifications. In some embodiments, the modified phosphodiester groups that link consecutive ribose nucleosides have been shown to enhance the stability and in vivo bioavailability of siRNAs The non-ester groups (—OH, ═O) of the phosphodiester linkage can be replaced with sulfur, boron, or acetate to give phosphorothioate, boranophosphate, and phosphonoacetate linkages. In addition, substituting the phosphodiester group with a phosphotriester can facilitate cellular uptake of siRNAs and retention on serum components by eliminating their negative charge. In some embodiments, the siRNA molecules have sugar modifications. In some embodiments, the sugars are deprotonated (reaction catalyzed by exo- and endonucleases) whereby the 2′-hydroxyl can act as a nucleophile and attack the adjacent phosphorous in the phosphodiester bond. Such alternatives include 2′-O-methyl, 2′-O-methoxyethyl, and 2′-fluoro modifications.

In some embodiments, the siRNA molecules have base modifications. In some embodiments, the bases can be substituted with modified bases such as pseudouridine, 5′-methylcytidine, N6-methyladenosine, inosine, and N7-methylguanosine.

In some embodiments, the siRNA molecules are conjugated to lipids. Lipids can be conjugated to the 5′ or 3′ termini of siRNA to improve their in vivo bioavailability by allowing them to associate with serum lipoproteins. Representative lipids include, but are not limited to, cholesterol and vitamin E, and fatty acids, such as palmitate and tocopherol.

In some embodiments, a representative siRNA has the following formula:

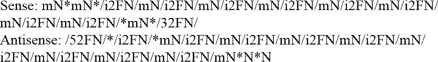

wherein: “N” is the base; “2F” is a 2′-F modification; “m” is a 2′-O-methyl modification, “I” is an internal base; and “*” is a phosphorothioate backbone linkage.

In any of the embodiments described herein, the inhibitory nucleic acid molecules may be administered, for example, as one to two hour i.v. infusions or s.c. injections. In any of the embodiments described herein, the inhibitory nucleic acid molecules may be administered at dose levels that range from about 50 mg to about 900 mg, from about 100 mg to about 800 mg, from about 150 mg to about 700 mg, or from about 175 to about 640 mg (2.5 to 9.14 mg/kg; 92.5 to 338 mg/m² - based on an assumption of a body weight of 70 kg and a conversion of mg/kg to mg/m² dose levels based on a mg/kg dose multiplier value of 37 for humans).

The present disclosure also provides vectors comprising any one or more of the inhibitory nucleic acid molecules. In some embodiments, the vectors comprise any one or more of the inhibitory nucleic acid molecules and a heterologous nucleic acid. The vectors can be viral or nonviral vectors capable of transporting a nucleic acid molecule. In some embodiments, the vector is a plasmid or cosmid (such as, for example, a circular double-stranded DNA into which additional DNA segments can be ligated). In some embodiments, the vector is a viral vector, wherein additional DNA segments can be ligated into the viral genome. Expression vectors include, but are not limited to, plasmids, cosmids, retroviruses, adenoviruses, adeno-associated viruses (AAV), plant viruses such as cauliflower mosaic virus and tobacco mosaic virus, yeast artificial chromosomes (YACs), Epstein-Barr (EBV)-derived episomes, and other expression vectors known in the art.

The present disclosure also provides compositions comprising any one or more of the inhibitory nucleic acid molecules. In some embodiments, the composition is a pharmaceutical composition. In some embodiments, the compositions comprise a carrier and/or excipient. Examples of carriers include, but are not limited to, poly(lactic acid) (PLA) microspheres, poly(D,L-lactic-coglycolic-acid) (PLGA) microspheres, liposomes, micelles, inverse micelles, lipid cochleates, and lipid microtubules. A carrier may comprise a buffered salt solution such as PBS, HBSS, etc.

In some embodiments, the TCL1A antagonist comprises a nuclease agent that induces one or more nicks or double-strand breaks at a recognition sequence(s) or a DNA-binding protein that binds to a recognition sequence within a TCL1A genomic nucleic acid molecule. The recognition sequence can be located within a coding region of the TCL1A gene, or within regulatory regions that influence the expression of the gene. A recognition sequence of the DNA-binding protein or nuclease agent can be located in an intron, an exon, a promoter, an enhancer, a regulatory region, or any non-protein coding region. The recognition sequence can include or be proximate to the start codon of the TCL1A gene. For example, the recognition sequence can be located about 10, about 20, about 30, about 40, about 50, about 100, about 200, about 300, about 400, about 500, or about 1,000 nucleotides from the start codon. As another example, two or more nuclease agents can be used, each targeting a nuclease recognition sequence including or proximate to the start codon. As another example, two nuclease agents can be used, one targeting a nuclease recognition sequence including or proximate to the start codon, and one targeting a nuclease recognition sequence including or proximate to the stop codon, wherein cleavage by the nuclease agents can result in deletion of the coding region between the two nuclease recognition sequences. Any nuclease agent that induces a nick or double-strand break into a desired recognition sequence can be used in the methods and compositions disclosed herein. Any DNA-binding protein that binds to a desired recognition sequence can be used in the methods and compositions disclosed herein.

Suitable nuclease agents and DNA-binding proteins for use herein include, but are not limited to, zinc finger protein or zinc finger nuclease (ZFN) pair, Transcription Activator-Like Effector (TALE) protein or Transcription Activator-Like Effector Nuclease (TALEN), or Clustered Regularly Interspersed Short Palindromic Repeats (CRISPR)/CRISPR-associated (Cas) systems. The length of the recognition sequence can vary, and includes, for example, recognition sequences that are about 30-36 bp for a zinc finger protein or ZFN pair, about 15-18 bp for each ZFN, about 36 bp for a TALE protein or TALEN, and about 20 bp for a CRISPR/Cas guide RNA.

In some embodiments, CRISPR/Cas systems can be used to modify the TCL1A genomic nucleic acid molecule within a cell. The methods and compositions disclosed herein can employ CRISPR-Cas systems by utilizing CRISPR complexes (comprising a guide RNA (gRNA) complexed with a Cas protein) for site-directed cleavage of TCL1A nucleic acid molecules.

Cas proteins generally comprise at least one RNA recognition or binding domain that can interact with gRNAs. Cas proteins can also comprise nuclease domains (such as, for example, DNase or RNase domains), DNA binding domains, helicase domains, protein-protein interaction domains, dimerization domains, and other domains. Suitable Cas proteins include, for example, a wild type Cas9 protein and a wild type Cpf1 protein (such as, for example, FnCpf1). A Cas protein can have full cleavage activity to create a double-strand break in a TCL1A genomic nucleic acid molecule or it can be a nickase that creates a single-strand break in a TCL1A genomic nucleic acid molecule. Additional examples of Cas proteins include, but are not limited to, Cas1, Cas1B, Cas2, Cas3, Cas4, Cas5, Cas5e (CasD), Cas6, Cas6e, Cas6f, Cas7, Cas8a1, Cas8a2, Cas8b, Cas8c, Cas9 (Csn1 or Csx12), Cas10, Cas10d, CasF, CasG, CasH, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cu1966, and homologs or modified versions thereof. Cas proteins can also be operably linked to heterologous polypeptides as fusion proteins. For example, a Cas protein can be fused to a cleavage domain, an epigenetic modification domain, a transcriptional activation domain, or a transcriptional repressor domain. Cas proteins can be provided in any form. For example, a Cas protein can be provided in the form of a protein, such as a Cas protein complexed with a gRNA. Alternately, a Cas protein can be provided in the form of a nucleic acid molecule encoding the Cas protein, such as an RNA or DNA.

In some embodiments, targeted genetic modifications of TCL1A genomic nucleic acid molecules can be generated by contacting a cell with a Cas protein and one or more gRNAs that hybridize to one or more gRNA recognition sequences within a target genomic locus in the TCL1A genomic nucleic acid molecule. For example, an TCL1A gRNA recognition sequence can be located within a region of SEQ ID NO:1. The gRNA recognition sequence can include or be proximate to the start codon of a TCL1A genomic nucleic acid molecule or the stop codon of a TCL1A genomic nucleic acid molecule. For example, the gRNA recognition sequence can be located from about 10, from about 20, from about 30, from about 40, from about 50, from about 100, from about 200, from about 300, from about 400, from about 500, or from about 1,000 nucleotides of the start codon or the stop codon.

The gRNA recognition sequences within a target genomic locus in a TCL1A genomic nucleic acid molecule are located near a Protospacer Adjacent Motif (PAM) sequence, which is a 2-6 base pair DNA sequence immediately following the DNA sequence targeted by the Cas9 nuclease. The canonical PAM is the sequence 5′-NGG-3′ where “N” is any nucleobase followed by two guanine (“G”) nucleobases. gRNAs can transport Cas9 to anywhere in the genome for gene editing, but no editing can occur at any site other than one at which Cas9 recognizes PAM. In addition, 5′-NGA-3′ can be a highly efficient non-canonical PAM for human cells. Generally, the PAM is about 2-6 nucleotides downstream of the DNA sequence targeted by the gRNA. The PAM can flank the gRNA recognition sequence. In some embodiments, the gRNA recognition sequence can be flanked on the 3′ end by the PAM. In some embodiments, the gRNA recognition sequence can be flanked on the 5′ end by the PAM. For example, the cleavage site of Cas proteins can be about 1 to about 10, about 2 to about 5 base pairs, or three base pairs upstream or downstream of the PAM sequence. In some embodiments (such as when Cas9 from S. pyogenes or a closely related Cas9 is used), the PAM sequence of the non-complementary strand can be 5′-NGG-3′, where N is any DNA nucleotide and is immediately 3′ of the gRNA recognition sequence of the non-complementary strand of the target DNA. As such, the PAM sequence of the complementary strand would be 5′-CCN-3′, where N is any DNA nucleotide and is immediately 5′ of the gRNA recognition sequence of the complementary strand of the target DNA.

A gRNA is an RNA molecule that binds to a Cas protein and targets the Cas protein to a specific location within a TCL1A genomic nucleic acid molecule. An exemplary gRNA is a gRNA effective to direct a Cas enzyme to bind to or cleave a TCL1A genomic nucleic acid molecule, wherein the gRNA comprises a DNA-targeting segment that hybridizes to a gRNA recognition sequence within the TCL1A genomic nucleic acid molecule. Exemplary gRNAs comprise a DNA-targeting segment that hybridizes to a gRNA recognition sequence present within a TCL1A genomic nucleic acid molecule that includes or is proximate to the start codon or the stop codon. For example, a gRNA can be selected such that it hybridizes to a gRNA recognition sequence that is located from about 5, from about 10, from about 15, from about 20, from about 25, from about 30, from about 35, from about 40, from about 45, from about 50, from about 100, from about 200, from about 300, from about 400, from about 500, or from about 1,000 nucleotides of the start codon or located from about 5, from about 10, from about 15, from about 20, from about 25, from about 30, from about 35, from about 40, from about 45, from about 50, from about 100, from about 200, from about 300, from about 400, from about 500, or from about 1,000 nucleotides of the stop codon. Suitable gRNAs can comprise from about 17 to about 25 nucleotides, from about 17 to about 23 nucleotides, from about 18 to about 22 nucleotides, or from about 19 to about 21 nucleotides. In some embodiments, the gRNAs can comprise 20 nucleotides.

The Cas protein and the gRNA form a complex, and the Cas protein cleaves the target TCL1A genomic nucleic acid molecule. The Cas protein can cleave the nucleic acid molecule at a site within or outside of the nucleic acid sequence present in the target TCL1A genomic nucleic acid molecule to which the DNA-targeting segment of a gRNA will bind. For example, formation of a CRISPR complex (comprising a gRNA hybridized to a gRNA recognition sequence and complexed with a Cas protein) can result in cleavage of one or both strands in or near (such as, for example, within 1, 2, 3, 4, 5, 6, 7, 8, 9, 10, 20, 50, or more base pairs from) the nucleic acid sequence present in the TCL1A genomic nucleic acid molecule to which a DNA-targeting segment of a gRNA will bind.

Such methods can result, for example, in a TCL1A genomic nucleic acid molecule in which a region of SEQ ID NO:1 is disrupted, the start codon is disrupted, the stop codon is disrupted, or the coding sequence is disrupted or deleted. Optionally, the cell can be further contacted with one or more additional gRNAs that hybridize to additional gRNA recognition sequences within the target genomic locus in the TCL1A genomic nucleic acid molecule. By contacting the cell with one or more additional gRNAs (such as, for example, a second gRNA that hybridizes to a second gRNA recognition sequence), cleavage by the Cas protein can create two or more double-strand breaks or two or more single-strand breaks.

In some embodiments, the methods further comprising detecting the presence or absence of a TCL1A variant nucleic acid molecule in a biological sample from the subject. In some embodiments, the TCL1A variant nucleic acid molecule is a missense variant, splice-site variant, a stop-gain variant, a start-loss variant, a stop-loss variant, a frameshift variant, or an in-frame indel variant, or a variant that encodes a truncated predicted loss-of-function polypeptide. In some embodiments, the TCL1A variant nucleic acid molecule is a missense variant. In some embodiments, the TCL1A variant nucleic acid molecule is a splice-site variant. In some embodiments, the TCL1A variant nucleic acid molecule is a stop-gain variant. In some embodiments, the TCL1A variant nucleic acid molecule is a start-loss variant. In some embodiments, the TCL1A variant nucleic acid molecule is a stop-loss variant. In some embodiments, the TCL1A variant nucleic acid molecule is a frameshift variant. In some embodiments, the TCL1A variant nucleic acid molecule is an in-frame indel variant. In some embodiments, the TCL1A variant nucleic acid molecule is a variant that encodes a truncated predicted loss-of-function polypeptide. In some embodiments, the TCL1A variant nucleic acid molecule comprises the rs2296311, rs2887399, or rs11846938 single nucleotide polymorphism. In some embodiments, the TCL1A variant nucleic acid molecule comprises the rs2296311 single nucleotide polymorphism. In some embodiments, the TCL1A variant nucleic acid molecule comprises the rs2887399 single nucleotide polymorphism. In some embodiments, the TCL1A variant nucleic acid molecule comprises the rs11846938 single nucleotide polymorphism.

The nucleotide sequence of a genomic wild-type TCL1A is set forth in SEQ ID NO:1 (GRCh38/hg38 chr14:95709467-95714696; ENSG00000100721.11 plus 500 bp 5′ and 3′; 5,230 bp).

The rs2887399 variant is located at chr14:95714358 (GRCh38.p13) and is a 2 KB upstream variant. Before this disclosure, the rs2887399 variant was of unknown clinical significance. The nucleotide sequence set forth in SEQ ID NO:2 comprises the nucleotide sequence of rs2887399 TCL1A with a C->A at position 339. In some embodiments, the rs2887399 variant can be a germline rs2887399 variant. In some embodiments, the rs2887399 variant can be a somatic rs2887399 variant.

The rs11846938 variant is located at chr14:95714348 (GRCh38.p13) and is a 2 KB upstream variant. Before this disclosure, the rs11846938 variant was of unknown clinical significance. The nucleotide sequence set forth in SEQ ID NO:3 comprises the nucleotide sequence of rs11846938 TCL1A with an A->C at position 349. In some embodiments, the rs11846938 variant can be a germline rs11846938 variant. In some embodiments, the rs11846938 variant can be a somatic rs11846938 variant.

The rs2296311 variant is located at chr14:95711836 (GRCh38.p13) and is an intron variant. Before this disclosure, the rs2296311 variant was of unknown clinical significance. The nucleotide sequence set forth in SEQ ID NO:4 comprises the nucleotide sequence of rs2296311 TCL1A with C->T at position 2,861. In some embodiments, the rs2296311 variant can be a germline rs2296311 variant. In some embodiments, the rs2296311 variant can be a somatic rs2296311 variant.

The biological sample can be derived from any cell, tissue, or biological fluid from the subject. The biological sample may comprise any clinically relevant tissue, such as lung tissue or lung cells, such as from a biopsy, a fine needle aspirate, or a sample of bodily fluid, such as blood, gingival crevicular fluid, plasma, serum, lymph, ascitic fluid, cystic fluid, or urine. In some cases, the sample comprises a buccal swab. The biological sample used in the methods disclosed herein can vary based on the assay format, nature of the detection method, and the tissues, cells, or extracts that are used as the sample.

In some embodiments, the methods further comprise administering a therapeutic agent that treats, prevents, or reduces development of CHIP in a standard dosage amount to a subject wherein a TCL1A variant nucleic acid molecule is absent from the biological sample. In some embodiments, the methods further comprise administering a therapeutic agent that treats, prevents, or reduces development of CHIP in a standard dosage amount to a subject wherein a TCL1A variant nucleic acid molecule is present in the biological sample. In some embodiments, the methods further comprise administering a therapeutic agent that treats, prevents, or reduces development of CHIP in a dosage amount that is the same as, greater than, or less than a standard dosage amount to a subject that is heterozygous for a TCL1A variant nucleic acid molecule. In some embodiments, the methods further comprise administering a therapeutic agent that treats, prevents, or reduces development of CHIP in a dosage amount that is the same as, less than, or greater than a standard dosage amount to a subject that is heterozygous for a TCL1A variant nucleic acid molecule.

The terms “treat”, “treating”, and “treatment” and “prevent”, “preventing”, and “prevention” as used herein, refer to eliciting the desired biological response, such as a therapeutic and prophylactic effect, respectively. In some embodiments, a therapeutic effect comprises one or more of a decrease/reduction in CHIP, a decrease/reduction in the severity of CHIP (such as, for example, a reduction or inhibition of development of CHIP), a decrease/reduction in symptoms and CHIP-related effects, delaying the onset of symptoms and CHIP-related effects, reducing the severity of symptoms of c CHIP-related effects, reducing the number of symptoms and CHIP-related effects, reducing the latency of symptoms and CHIP-related effects, an amelioration of symptoms and CHIP-related effects, reducing secondary symptoms, reducing secondary infections, preventing relapse to CHIP, decreasing the number or frequency of relapse episodes, increasing latency between symptomatic episodes, increasing time to sustained progression, speeding recovery, or increasing efficacy of or decreasing resistance to alternative therapeutics, and/or an increased survival time of the affected host animal, following administration of the agent or composition comprising the agent. A prophylactic effect may comprise a complete or partial avoidance/inhibition or a delay of CHIP development/progression (such as, for example, a complete or partial avoidance/inhibition or a delay), and an increased survival time of the affected host animal, following administration of a therapeutic protocol. Treatment of CHIP encompasses the treatment of a subject already diagnosed as having any form of CHIP at any clinical stage or manifestation, the delay of the onset or evolution or aggravation or deterioration of the symptoms or signs of CHIP, and/or preventing and/or reducing the severity of CHIP.

Alteration-specific polymerase chain reaction techniques can be used to detect mutations such as SNPs in a nucleic acid sequence. Alteration-specific primers can be used because the DNA polymerase will not extend when a mismatch with the template is present.

In some embodiments, the assay comprises RNA sequencing (RNA-Seq). In some embodiments, the assays also comprise reverse transcribing mRNA into cDNA, such as by the reverse transcriptase polymerase chain reaction (RT-PCR).

Illustrative examples of nucleic acid sequencing techniques include, but are not limited to, chain terminator (Sanger) sequencing and dye terminator sequencing. Other methods involve nucleic acid hybridization methods other than sequencing, including using labeled primers or probes directed against purified DNA, amplified DNA, and fixed cell preparations (fluorescence in situ hybridization (FISH)). In some methods, a target nucleic acid molecule may be amplified prior to or simultaneous with detection. Illustrative examples of nucleic acid amplification techniques include, but are not limited to, polymerase chain reaction (PCR), ligase chain reaction (LCR), strand displacement amplification (SDA), and nucleic acid sequence based amplification (NASBA). Other methods include, but are not limited to, ligase chain reaction, strand displacement amplification, and thermophilic SDA (tSDA).

In hybridization techniques, stringent conditions can be employed such that a probe or primer will specifically hybridize to its target. In some embodiments, a polynucleotide primer or probe under stringent conditions will hybridize to its target sequence to a detectably greater degree than to other non-target sequences, such as, at least 2-fold, at least 3-fold, at least 4-fold, or more over background, including over 10-fold over background. In some embodiments, a polynucleotide primer or probe under stringent conditions will hybridize to its target nucleotide sequence to a detectably greater degree than to other nucleotide sequences by at least 2-fold. In some embodiments, a polynucleotide primer or probe under stringent conditions will hybridize to its target nucleotide sequence to a detectably greater degree than to other nucleotide sequences by at least 3-fold. In some embodiments, a polynucleotide primer or probe under stringent conditions will hybridize to its target nucleotide sequence to a detectably greater degree than to other nucleotide sequences by at least 4-fold. In some embodiments, a polynucleotide primer or probe under stringent conditions will hybridize to its target nucleotide sequence to a detectably greater degree than to other nucleotide sequences by over 10-fold over background. Stringent conditions are sequence-dependent and will be different in different circumstances.

Appropriate stringency conditions which promote DNA hybridization, for example, 6X sodium chloride/sodium citrate (SSC) at about 45° C., followed by a wash of 2X SSC at 50° C., are known or can be found in Current Protocols in Molecular Biology, John Wiley & Sons, N.Y. (1989), 6.3.1-6.3.6. Typically, stringent conditions for hybridization and detection will be those in which the salt concentration is less than about 1.5 M Na⁺ ion, typically about 0.01 to 1.0 M Na⁺ ion concentration (or other salts) at pH 7.0 to 8.3 and the temperature is at least about 30° C. for short probes (such as, for example, 10 to 50 nucleotides) and at least about 60° C. for longer probes (such as, for example, greater than 50 nucleotides). Stringent conditions may also be achieved with the addition of destabilizing agents such as formamide. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 200, at least about 300, at least about 400, at least about 500, at least about 600, at least about 700, at least about 800, at least about 900, at least about 1000, at least about 2000, at least about 3000, at least about 4000, or at least about 5000 nucleotides. In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, or at least about 25 nucleotides. In some embodiments, the isolated nucleic acid molecules comprise or consist of at least about 18 nucleotides. In some embodiments, the isolated nucleic acid molecules comprise or consists of at least about 15 nucleotides. In some embodiments, the isolated nucleic acid molecules consist of or comprise from about 10 to about 35, from about 10 to about 30, from about 10 to about 25, from about 12 to about 30, from about 12 to about 28, from about 12 to about 24, from about 15 to about 30, from about 15 to about 25, from about 18 to about 30, from about 18 to about 25, from about 18 to about 24, or from about 18 to about 22 nucleotides. In some embodiments, the isolated nucleic acid molecules consist of or comprise from about 18 to about 30 nucleotides. In some embodiments, the isolated nucleic acid molecules comprise or consist of at least about 15 nucleotides to at least about 35 nucleotides.

In some embodiments, the alteration-specific probes and alteration-specific primers comprise DNA. In some embodiments, the alteration-specific probes and alteration-specific primers comprise RNA.

In some embodiments, the probes and primers described herein (including alteration-specific probes and alteration-specific primers) have a nucleotide sequence that specifically hybridizes to any of the nucleic acid molecules disclosed herein, or the complement thereof. In some embodiments, the probes and primers specifically hybridize to any of the nucleic acid molecules disclosed herein under stringent conditions.

In some embodiments, the primers, including alteration-specific primers, can be used in second generation sequencing or high throughput sequencing. In some instances, the primers, including alteration-specific primers, can be modified. In particular, the primers can comprise various modifications that are used at different steps of, for example, Massive Parallel Signature Sequencing (MPSS), Polony sequencing, and 454 Pyrosequencing. Modified primers can be used at several steps of the process, including biotinylated primers in the cloning step and fluorescently labeled primers used at the bead loading step and detection step. Polony sequencing is generally performed using a paired-end tags library wherein each molecule of DNA template is about 135 bp in length. Biotinylated primers are used at the bead loading step and emulsion PCR. Fluorescently labeled degenerate nonamer oligonucleotides are used at the detection step. An adaptor can contain a 5′-biotin tag for immobilization of the DNA library onto streptavidin-coated beads.

In some embodiments, the probes (such as, for example, an alteration-specific probe) comprise a label. In some embodiments, the label is a fluorescent label, a radiolabel, or biotin.

The present disclosure also provides supports comprising a substrate to which any one or more of the probes disclosed herein is attached. Solid supports are solid-state substrates or supports with which molecules, such as any of the probes disclosed herein, can be associated. A form of solid support is an array. Another form of solid support is an array detector. An array detector is a solid support to which multiple different probes have been coupled in an array, grid, or other organized pattern. A form for a solid-state substrate is a microtiter dish, such as a standard 96-well type. In some embodiments, a multiwell glass slide can be employed that normally contains one array per well.

The nucleic acid molecules can be from any organism. For example, the nucleic acid molecules can be human or an ortholog from another organism, such as a non-human mammal, a rodent, a mouse, or a rat. It is understood that gene sequences within a population can vary due to polymorphisms such as single-nucleotide polymorphisms. The examples provided herein are only exemplary sequences. Other sequences are also possible.

The isolated nucleic acid molecules disclosed herein can comprise RNA, DNA, or both RNA and DNA. The isolated nucleic acid molecules can also be linked or fused to a heterologous nucleic acid sequence, such as in a vector, or a heterologous label. For example, the isolated nucleic acid molecules disclosed herein can be within a vector or as an exogenous donor sequence comprising the isolated nucleic acid molecule and a heterologous nucleic acid sequence. The isolated nucleic acid molecules can also be linked or fused to a heterologous label. The label can be directly detectable (such as, for example, fluorophore) or indirectly detectable (such as, for example, hapten, enzyme, or fluorophore quencher). Such labels can be detectable by spectroscopic, photochemical, biochemical, immunochemical, or chemical means. Such labels include, for example, radiolabels, pigments, dyes, chromogens, spin labels, and fluorescent labels. The label can also be, for example, a chemiluminescent substance; a metal-containing substance; or an enzyme, where there occurs an enzyme-dependent secondary generation of signal. The term “label” can also refer to a “tag” or hapten that can bind selectively to a conjugated molecule such that the conjugated molecule, when added subsequently along with a substrate, is used to generate a detectable signal. For example, biotin can be used as a tag along with an avidin or streptavidin conjugate of horseradish peroxidate (HRP) to bind to the tag, and examined using a calorimetric substrate (such as, for example, tetramethylbenzidine (TMB)) or a fluorogenic substrate to detect the presence of HRP. Exemplary labels that can be used as tags to facilitate purification include, but are not limited to, myc, HA, FLAG or 3XFLAG, 6Xhis or polyhistidine, glutathione-S-transferase (GST), maltose binding protein, an epitope tag, or the Fc portion of immunoglobulin. Numerous labels include, for example, particles, fluorophores, haptens, enzymes and their calorimetric, fluorogenic and chemiluminescent substrates and other labels.

The present disclosure also provides methods of identifying a subject having an increased risk of developing CHIP, wherein the subject comprises a DNMT3A-CHIP mutation. These methods comprise determining or having determined the presence or absence of a TCL1A variant nucleic acid molecule in a biological sample obtained from the subject. When the subject is TCL1A reference, then the subject has a decreased risk of developing CHIP compared to a subject that comprises the TCL1A variant nucleic acid molecule. When the subject is heterozygous or homozygous for the TCL1A variant nucleic acid molecule, then the subject has an increased risk of developing CHIP compared to a subject that is TCL1A reference. In some embodiments, when the subject is TCL1A reference, the subject is administered or continued to be administered a therapeutic agent that treats, prevents, or reduces development of CHIP in a standard dosage amount, and/or is administered a TCL1A antagonist. In some embodiments, when the subject is heterozygous for a TCL1A variant nucleic acid molecule, the subject is administered or continued to be administered a therapeutic agent that treats, prevents, or reduces development of CHIP in an amount that is the same as, greater than, or less than a standard dosage amount, and/or is administering a TCL1A antagonist. In some embodiments, when the subject is homozygous for a TCL1A variant nucleic acid molecule, the subject is administered or continued to be administered a therapeutic agent that treats, prevents, or reduces development of CHIP in an amount that is the same as, greater than, or less than a standard dosage amount.

The present disclosure also provides methods of identifying a subject having a decreased risk of developing CHIP, wherein the subject comprises a TET2-CHIP mutation. These methods comprise determining or having determined the presence or absence of a TCL1A variant nucleic acid molecule in a biological sample obtained from the subject. When the subject is TCL1A reference, then the subject has an increased risk of developing CHIP compared to a subject that comprises the TCL1A variant nucleic acid molecule. When the subject is heterozygous or homozygous for the TCL1A variant nucleic acid molecule, then the subject has a decreased risk of developing CHIP compared to a subject that is TCL1A reference. In some embodiments, when the subject is TCL1A reference, the subject is administered or continued to be administered a therapeutic agent that treats, prevents, or reduces development of CHIP in a standard dosage amount, and/or is administered a TCL1A antagonist. In some embodiments, when the subject is heterozygous for a TCL1A variant nucleic acid molecule, the subject is administered or continued to be administered a therapeutic agent that treats, prevents, or reduces development of CHIP in an amount that is the same as, greater than, or less than a standard dosage amount, and/or is administering a TCL1A antagonist. In some embodiments, when the subject is homozygous for a TCL1A variant nucleic acid molecule, the subject is administered or continued to be administered a therapeutic agent that treats, prevents, or reduces development of CHIP in an amount that is the same as, greater than, or less than a standard dosage amount.

The present disclosure also provides methods of identifying a subject having a decreased risk of developing CHIP, wherein the subject comprises an ASXL1-CHIP mutation. These methods comprise determining or having determined the presence or absence of a TCL1A variant nucleic acid molecule in a biological sample obtained from the subject. When the subject is TCL1A reference, then the subject has an increased risk of developing CHIP compared to a subject that comprises the TCL1A variant nucleic acid molecule. When the subject is heterozygous or homozygous for the TCL1A variant nucleic acid molecule, then the subject has a decreased risk of developing CHIP compared to a subject that is TCL1A reference. In some embodiments, when the subject is TCL1A reference, the subject is administered or continued to be administered a therapeutic agent that treats, prevents, or reduces development of CHIP in a standard dosage amount, and/or is administered a TCL1A antagonist. In some embodiments, when the subject is heterozygous for a TCL1A variant nucleic acid molecule, the subject is administered or continued to be administered a therapeutic agent that treats, prevents, or reduces development of CHIP in an amount that is the same as, greater than, or less than a standard dosage amount, and/or is administering a TCL1A antagonist. In some embodiments, when the subject is homozygous for a TCL1A variant nucleic acid molecule, the subject is administered or continued to be administered a therapeutic agent that treats, prevents, or reduces development of CHIP in an amount that is the same as, greater than, or less than a standard dosage amount.

In any of the embodiments described herein, the TCL1A variant nucleic acid molecule can be any TCL1A variant nucleic acid molecule described herein.

In any of the embodiments described herein, the subject can have or be at risk of developing a hematologic cancer, a myeloid neoplasia, a lymphoid neoplasia, an atherosclerotic cardiovascular disease, a coronary heart disease, a myocardial infarction, or severe calcified aortic valve stenosis.

In any of the embodiments described herein, the TCL1A antagonist can be any of the TCL1A antagonists described herein.

The present disclosure also provides methods of identifying a subject having an increased risk of developing a solid tumor. These methods comprise determining or having determined the presence or absence of at least one CHIP somatic mutation at a high variant allele fraction (VAF) in a biological sample obtained from the subject. When the subject has a high VAF of at least one CHIP somatic mutation, then the subject has an increased risk of developing the solid tumor. When the subject does not have a high VAF of at least one CHIP somatic mutation, then the subject does not have an increased risk of developing the solid tumor.

In some embodiments, the subject comprises a DNMT3A-CHIP somatic mutation. In some embodiments, the subject comprises a TET2-CHIP somatic mutation. In some embodiments, the subject comprises an ASXL1-CHIP somatic mutation.

In some embodiments, the solid tumor is a lung cancer tumor. In some embodiments, the solid tumor is a prostate cancer tumor. In some embodiments, the solid tumor is a breast cancer tumor.

In some embodiments, the VAF is greater than 5%. In some embodiments, the VAF is greater than 6%. In some embodiments, the VAF is greater than 7%. In some embodiments, the VAF is greater than 8%. In some embodiments, the VAF is greater than 9%. In some embodiments, the VAF is greater than 10%.

The present disclosure also provides methods of identifying a subject having an increased risk of developing a blood cancer. These methods comprise determining or having determined the presence or absence of at least one CHIP somatic mutation at a high VAF in a biological sample obtained from the subject. When the subject has a high VAF of at least one CHIP somatic mutation, then the subject has an increased risk of developing the blood cancer. When the subject does not have a high VAF of at least one CHIP somatic mutation, then the subject does not have an increased risk of developing the blood cancer.

In some embodiments, the methods further comprises determining or having determined the presence or absence of at least one TET2-CHIP somatic mutation, at least one ASXL1-CHIP somatic mutation, and/or at least one DNMT3A-CHIP somatic mutation in the biological sample obtained from the subject. When the subject has at least one TET2-CHIP somatic mutation and/or at least one ASXL1-CHIP somatic mutation, and the subject is a smoker or a non-smoker, then the subject has an increased risk of developing the blood cancer. When the subject has at least one DNMT3A-CHIP somatic mutation, and the subject is a non-smoker, then the subject has an increased risk of developing the blood cancer. When the subject has at least one DNMT3A-CHIP somatic mutation, and the subject is a smoker, then the subject does not have an increased risk of developing the blood cancer compared to a non-smoker.

In some embodiments, the VAF is greater than 5%. In some embodiments, the VAF is greater than 6%. In some embodiments, the VAF is greater than 7%. In some embodiments, the VAF is greater than 8%. In some embodiments, the VAF is greater than 9%. In some embodiments, the VAF is greater than 10%.

In any of the embodiments described herein, representative DNMT3A somatic mutations include, but are not limited to:

Lengthy table referenced here
US20230340598A1-20231026-T00001
Please refer to the end of the specification for access instructions.

Lengthy table referenced here
US20230340598A1-20231026-T00002
Please refer to the end of the specification for access instructions.

Lengthy table referenced here
US20230340598A1-20231026-T00003
Please refer to the end of the specification for access instructions.

Percent identity (or percent complementarity) between particular stretches of nucleotide sequences within nucleic acid molecules or amino acid sequences within polypeptides can be determined routinely using BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489). Herein, if reference is made to percent sequence identity, the higher percentages of sequence identity are preferred over the lower ones.

As used herein, the phrase “corresponding to” or grammatical variations thereof when used in the context of the numbering of a particular nucleotide or nucleotide sequence or position refers to the numbering of a specified reference sequence when the particular nucleotide or nucleotide sequence is compared to a reference sequence (such as, for example, SEQ ID NO:1). In other words, the residue (such as, for example, nucleotide or amino acid) number or residue (such as, for example, nucleotide or amino acid) position of a particular polymer is designated with respect to the reference sequence rather than by the actual numerical position of the residue within the particular nucleotide or nucleotide sequence. For example, a particular nucleotide sequence can be aligned to a reference sequence by introducing gaps to optimize residue matches between the two sequences. In these cases, although the gaps are present, the numbering of the residue in the particular nucleotide or nucleotide sequence is made with respect to the reference sequence to which it has been aligned.

The nucleotide and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. The amino acid sequence follows the standard convention of beginning at the amino terminus of the sequence and proceeding forward (i.e., from left to right in each line) to the carboxy terminus.

The present disclosure also provides methods of treating a subject having CHIP or at risk of developing CHIP, the methods comprising administering HSCs to the subject, wherein the HSCs have been treated ex vivo with one or more TCL1A antagonists. In some embodiments, the HSCs are obtained from the subject being treated and hence are autologous cells. In some embodiments, the HSCs are obtained from a different individual and hence are donor cells.

In such methods, a composition comprising HSCs is administered to a subject. Such methods are well known in the art. The HSCs are optionally, although not necessarily, purified (Klein et al., Bone Marrow Transplant., 2001, 28, 1023-9; Prince et al., Cytotherapy, 2002, 4, 137-45; Prince et al., Cytotherapy, 2002, 4, 147-55; Handgretinger et al., Bone Marrow Transplant., 2002, 29, 731-6; and Chou et al., Breast Cancer, 2005, 12, 178-88). HSCs can be obtained by harvesting from bone marrow or from peripheral blood. Bone marrow is generally aspirated from the posterior iliac crests while the donor is under either regional or general anesthesia. Additional bone marrow can be obtained from the anterior iliac crest. A dose of 1 × 10⁸ and 2 × 10⁸ marrow mononuclear cells per kilogram is generally considered desirable to establish transplantation. Bone marrow can be primed with granulocyte colony-stimulating factor (G-CSF; filgrastim) to increase the stem cell count. The HSCs which are employed may be fresh, frozen, or have been subject to prior culture. They may be fetal, neonate, or adult. HSCs may be obtained from fetal liver, bone marrow, blood, particularly G-CSF or GM-CSF mobilized peripheral blood, or any other conventional source. Cells for transplant are optionally isolated from other cells, where the manner in which the stem cells are separated from other cells of the hematopoietic or other lineage is not critical. If desired, a substantially homogeneous population of stem or progenitor cells may be obtained by selective isolation of cells free of markers associated with differentiated cells, while displaying epitopic characteristics associated with the stem cells. Modes of administration include, but are not limited to, intravascular, intracerebral, parenteral, intraperitoneal, intravenous, epidural, intraspinal, intrastemal, intra-articular, intra-synovial, intrathecal, intra-arterial, intracardiac, or intramuscular.

Examples of ex vivo HSC are reported in, for example: Naldini, Nat. Rev. Genet., 2011, 12, 301-315; Sasaki et al., Proc. Natl. Acad. Sci. USA, 2006, 103, 14537-14541; Petersen et al., Blood Adv., 2018, 2, 210-223; and

All patent documents, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the present disclosure can be used in combination with any other feature, step, element, embodiment, or aspect unless specifically indicated otherwise. Although the present disclosure has been described in some detail by way of illustration and example for purposes of clarity and understanding, it will be apparent that certain changes and modifications may be practiced within the scope of the appended claims.

The following examples are provided to describe the embodiments in greater detail. They are intended to illustrate, not to limit, the claimed embodiments. The following examples provide those of ordinary skill in the art with a disclosure and description of how the compounds, compositions, articles, devices and/or methods described herein are made and evaluated, and are intended to be purely exemplary and are not intended to limit the scope of any claims. Efforts have been made to ensure accuracy with respect to numbers (such as, for example, amounts, temperature, etc.), but some errors and deviations may be accounted for. Unless indicated otherwise, parts are parts by weight, temperature is in °C or is at ambient temperature, and pressure is at or near atmospheric.

### EXAMPLES

### Example 1: Materials and Methods

### Exome Sequencing and Variant Calling

Sample preparation and sequencing were carried out as previously described (Van Hout et al., Nature, 2020, 586, 749-756). Briefly, sequencing libraries were prepped using genomic DNA samples from the UKB Biobank, followed by multiplexed exome capture and sequencing. Sequencing was performed on the Illumina NovaSeq 6000 platform using S2 (first 50,000 samples) or S4 (all other samples) flow cells. Read mapping, variant calling, and quality control were carried out according to the SPB protocol described in Van Hout et al. (Nature, 2020, 586, 749-756), which included the mapping of reads to the hg38 reference genome with BWA MEM, the identification of small variants with WeCall, and the use of GLnexus to aggregate these files into joint-genotyped, multi-sample VCF files. Depth and allelic valance filters were then applied, and samples were filtered out if they showed disagreement between genetically-determined and reported sex (n=279), high rates of heterozygosity/contamination (VBID > 5%) (n=287), low sequence coverage, or genetically-determined sample duplication (n=721 total samples), or variant discordance between WES and genotyping platforms. While 454,787 samples were used to compile final VCF files, 413 did not have array data after QC, and so the total number of individuals used for association analysis was 454,374.

### Calling CHIP

To call CHIP carrier status, the MuTect2 (GATK v4.1.4.0) somatic caller (Benjamin et al., bioRxiv 861054, 2019, doi:10.1101/861054) was first used to generate a raw callset of somatic mutations across all individuals. This software aims to use mapping quality measures as well as allele frequency information to identify somatic mutations against a background of germline mutations and sequencing errors. Somatic population af_only data was used generated from gnomAD v2 as the reference source for germline allele frequency (Karczewski et al., Nature, 2020, 581, 434-443). A cohort-specific panel of normals (PON) was first generated, which was a set of per-site beta distributions, against which variants were modeled, a component of refining somatic likelihood assignment. Since CHIP was strongly associated with age, 100 random UKB samples aged 40 years old and 622 samples <18 years old samples in GHS to build cohort specific PONs were chosen from these samples. As an initial refinement step, 56 genes were selected that have been recurrently associated with CHIP in recent reports from the Broad (Jaiswal et al., New Eng. J. Med., 2017, 377, 111-121), the TOPMed Consortium (Bick et al., Nature, 2020, 586, 763-768), and the Integrative Cancer Genomics (IntOGen) project (Pich et al., Discovering the drivers of clonal hematopoiesis, bioRxiv, 2020), and filtered putative somatic mutations to those occurring within these genes. Additional QC was then performed, which comprised: i) removing multi-allelic somatic calls (i.e. requiring somatic genotypes to be called as “0/1”), ii) sequencing depth filters (DP>=20; AD>=3), iii) Jaiswal CHIP rules, iv) removing sites filtered as panel of normal by Mutect2, v) removing indels flagged by the MuTect2 position filter, vi) removing sites within homopolymer runs with AD<10|AF<0.08, vii) excluding missense variants in CBL and TET2 that failed AB, viii) removing high median AF and MAF variants, ix) excluding non-expanding mutations, x) removing variants with excess C>A or G>T at Low AD, xi) removing variants flagged as having F1R2/F2R1 strand bias, and xii) removing samples with excess calls (>10). Given that >90% of mutations belonged to 23 recurrent CHIP-associated genes, variants were restricted to those that occurred within these genes as a final step in order to maximize the specificity of the callset. The final CHIP callset comprised 29,669 CHIP mutations across 27,331 unique individuals from UK Biobank, and 14,766 CHIP mutations across 12,877 unique individuals from GHS. Variant allele fraction (VAF) was calculated using AD/AD+RD. *DP=Total depth; AD=Alternate allele depth; RD=Reference allele depth; AF=Allele frequency

### Defining CHIP and Mosaic Phenotypes

CHIP phenotypes were derived based on the mutation callset, whereas mosaic chromosomal alteration (mCA) phenotypes were derived based on previously published mCA calls from the UKB Biobank (Zekavat et al., Nature Med., 2021, 27, 1012-1024; Thompson et al., Nature, 2019, 575, 652-657; Loh et al., Nature, 2018, 559, 350-355). First, ICD codes were used to exclude 3,596 samples from UK Biobank and 1,222 samples from GHS that had a diagnosis of blood cancer prior to sample collection. 13,004 individuals from GHS were also excluded whose DNA samples were collected from saliva as opposed to blood. Multiple CHIP and mosaic phenotypes were then defined on the basis of whether carriers did (inclusive) or did not (exclusive) have other somatic phenotypes. For example, individuals with at least one CHIP mutation in the callset were defined as carriers for a CHIP_inclusive phenotype, whereas anyone with a CHIP mutation as well as an identified mCA was removed from this inclusive phenotype in order to define a CHIP_exclusive phenotype (21,587 cases and 364,072 controls). The association analysis with CHIP used this CHIP_inclusive phenotype, which included 26,734 cases and 364,073 controls in UK Biobank, and 12,480 cases and 148,849 controls in GHS. mLOY carriers were defined as male individuals with a Y chromosome mCA in the UK Biobank mCA callset that had copy change status of loss or unknown, mLOX as individuals with an X chromosome mCA in the UK Biobank mCA callset that had copy change status of loss or unknown, and mCAaut carriers as individuals with autosomal mCAs. These inclusive phenotypes were then refined to define exclusive versions, with mLOY_exclusive comprising carriers with no X chromosome or autosomal mCAs (36,187 cases and 151,161), mLOX_exclusive comprising carriers with no Y chromosome or autosomal mCAs (10,743 cases and 364,072), and mCAaut_exclusive comprising carriers with no Y or X chromosomal alterations of any kind (11,154 cases and 364,072 controls). These exclusive phenotypes were used for all analyses comparing CHIP with mosaic phenotypes, as this approach facilitated the generation of four non-overlapping phenotypes (i.e. CHIP, mLOY, mLOX, and mCAaut) that could be compared and contrasted. CHIP gene-specific phenotypes were also defined by choosing carriers as those with mutations in the callset from a specific gene and no mutations in any other of the 23 CHIP genes that defined the callset. For example, CHIP-DNMT3A carriers were those with >=1 somatic mutations in the callset within the DNMT3A gene, and no mutations in our callset in any of the other 23 CHIP genes were used for the final callset definition. Given this definition, the CHIP gene-specific phenotypes were “exclusive” with regard to CHIP mutation subtype, but with regard to mosaic phenotypes, these CHIP gene specific phenotypes were inclusive (similar to the overall CHIP analysis).

### Genetic Association Analyses

To perform genetic association analyses, the genome-wide regression approach implemented in REGENIE (Mbatchou et al., Computationally efficient whole genome regression for quantitative and binary traits, bioRxiv, 2020) was used, as described in Backman et al. (Nature, 2021, 1-10, doi:10.1038/s41586-021-04103-z). Briefly, regressions were run separately for data derived from exome-sequencing as well as data derived from genetic imputation using TOPMed (Taliun et al., Nature, 2021, 590, 290-299), and results were combined across these data sources for downstream analysis. Step 1 of REGENIE uses genetic data to predict individual values for the trait of interest (i.e., a PRS), which is then used as a covariate in step 2 to adjust for population structure and other potential confounding. For step 1, variants were used from array data with a minor allele frequency (MAF) >1%, <10% missingness, Hardy-Weinberg equilibrium test P-value>10⁻¹⁵ and linkage disequilibrium (LD) pruning (1000 variant windows, 100 variant sliding windows and r²<0.9), and excluding any variants with high inter-chromosomal LD, in the major histocompatibility (MHC) region, or in regions of low complexity. For association analyses in step 2 of REGENIE, age, age², sex, and age-by-sex, were used with 10 ancestry-informative principal components (PCs) as covariates. For analyses involving exome data, also included as a covariate an indicator variable representing exome sequencing batch, and 20 PCs derived from the analysis of rare exomic variants (MAF between 2.6×10⁻⁵ and 0.01). Results were visualized and processed using in an house versions of the FUMA software (Watanabe et al., Nature Commun., 2017, 8, 1-11). Association analyses were performed separately for different continental ancestries defined based on the array data, as described in Backman et al. (Nature, 2021, 1-10, doi:10.1038/s41586-021-04103-z).

### Genetic Comparisons and Analyses

### Pairwise Mutational Analyses

Pairwise mutational enrichments across the UK Biobank and GHS CHIP callsets were calculated using Fishers’s Exact test among carriers with >=2 somatic CHIP mutations. For individuals with more than two CHIP mutations, the simplifying assumption of considering each mutation pair independently was made in the tested counts.

### Genetic Comparisons Between CHIP Subtypes

For pairwise comparisons between CHIP gene mutation subtypes, the union set of index SNPs (i.e. independent signals in genome-wide significant loci) was used from all of our CHIP and CHIP gene subtype associations. This resulted in 91 variants, which were used to compare effect sizes estimates between CHIP subtype pairs. Associations were estimated using linear regression, with the estimated effect size of variants in trait one as the dependent variable and the estimated effect size of variants in trait two as the independent variable. The ggplot2 package in R was also used to visualize all CHIP subtype comparisons together. Genetic correlations and trait heritability estimates were calculated using Idsc version 1.0.1 with annotation input version 2.2 (Finucane et al., Nature Genet., 2015, 47, 1228-1235).

### Pairwise Comparisons Between CHIP and Mosaic Phenotypes

For pairwise comparisons between CHIP and mLOY, mLOX, and mCAaut, the union set of index SNPs (i.e. independent signals in genome-wide significant loci) were used from all of our CHIP, CHIP gene subtype, and mosaic associations. This resulted in 341 variants, which were used to compare effect sizes estimates between phenotypic pairs. Pairwise associations were estimated using linear regression, with the estimated effect size of variants in trait one as the dependent variable and the estimated effect size of variants in trait two as the independent variable. Genetic correlations and trait heritability estimates were calculated using Idsc version 1.0.1 with annotation input version 2.2 (Finucane et al., Nature Genet., 2015, 47, 1228-1235).

### Phenotypic Associations With CHIP and CHIP-Subtypes

To test for known as well as potentially novel associations, tests were performed for association between the CHIP_inclusive phenotype and available binary (BT) and quantitative (QT) traits from the UK Biobank (V5) and the Geisinger Health System MyCode Community Health Initiative cohort (GHS). Given that they reflected generic (and often redundant) descriptions, phenotypes were excluded whose descriptions included any of the following text strings: ‘not elsewhere classified’, ‘Other’, ‘other’, ‘Encounter’, ‘Unspecified’, ‘unspecified’, ‘Abnormal’, ‘ Acquired’, ‘absence’, ‘not carried out’, ‘Personal history’, ‘personal history’. BTs were also filtered with a minimum of 100 cases and QTs with measurements on at least 5,000 individuals. This left 2,452 QTs and 13,101 QTs in UKB, and X and Y in GHS, which we used to performed univariate association analyses (chi-squared tests for BTs and Wilcoxon tests for QTs) to narrow down candidate phenotypes of interest. Phenotypes significantly associated with CHIP or CHIP gene subtypes after Bonferroni correction (calculated separately for each cohort and for BTs and QTs) were tested for association with CHIP or CHIP gene subtypes in a Firth logistic regression framework using age sex smoking and the first two genetically determined PCs as covariates. All results from these Firth logistic regression associations are presented in supplementary tables, and associations passing a Bonferroni significance threshold (among Firth logistic regression tests, and calculated separately for each cohort and for BTs and QTs) were considered significant. After filtering for redundant and non-specific phenotypes, the remaining significant phenotypes were included in visualizations.

### Longitudinal Analysis of Hematological and Oncological Risk Within Individuals With CHIP

Longitudinal survival analyses were performed using cox proportional hazard models (coxph function) as implemented in the survival r package. Given that CHIP was strongly correlated with age, and that time to event intervals will reflect older age periods for CHIP carriers, models with age as the longitudinal variable, with individuals being considered from the age of initial sequencing until the age when they had an event or their initial age + 13.5 years (the maximum time to event considered). This allowed for an implicit consideration of age within the proportion hazard models, and these models were established using the Surv function in the aforementioned survival package. All models included 10 genetically determined PCs as covariates, and all analyses excluded individuals genetically determined to be 3^{rd} degree relatives or closer. A variety of CHIP codings were used as variables in the models to test for potential differences between high/low VAF CHIP and/or CHIP subtypes. These were defined in the following way: i) CHIP was the overall CHIP phenotype, as described above in the GWAS/ExWAS analyses; ii) CHIP VAF10 was similar to CHIP, but restricted to individuals who had at least one CHIP mutation with VAF >= 0.10; iii) DNMT3A VAF10, TET2 VAF10, and ASXL1 VAF10 included individuals who had at least one identified somatic mutation in the respective gene with a VAF >= 0.1. Note that this definition varied from the one used in the GWAS/ExWAS, which required carriers to have mutations in the specified CHIP gene and no mutations in any other CHIP genes. Since mutational exclusivity in addition to the VAF >= 0.1 threshold would have required an individual to have a high VAF CHIP gene mutation but also not have any other mutations, which was not realistic and also significantly lowered sample size, this adjusted definition was chosen for the survival analyses; and iv) DNMT3A plus VAF10, which was limited to only those individuals with a DNMT3A mutation and a somatic mutation in at least one other CHIP gene. In other words, it represented a subset of the DNM3A VAF10 phenotype.

For the CAD analyses, sex, LDL, HDL, pack years, smoking status (current vs former), BMI, essential primary hypertension, and type 2 diabetes mellitus were included as covariates. We also excluded samples with any diagnosis of malignant blood cancer prior to sequencing (n = 3,596). Missing LDL and HDL values were median imputed, and individuals on cholesterol medication had their raw LDL values increased by a factor of 1/0.68, as this reflects the average expected reduction in LDL cholesterol levels. IL6R missense variant (rs2228145-C) genotypes were modeled dominantly (coded as 1 for carriers of any allele and 0 otherwise), and the effect of this allele was modeled in additive, interactive, and CHIP status stratified proportional hazard models. Models considering only the initial 50k UK Biobank individuals restricted to the samples reported by Bick et al. (Bick et al., Circulation, 2020, 141, 124-131). For visualization, base (Kaplan Meyer) survival curves (i.e. no covariates) were estimated using the survfit function in the aforementioned Survival package, and made plots using the ggsurvplot function from the survminer package.

For models of cancers, CAD, and overall survival risk tested using all CHIP carriers, high VAF (VAF >= 0.1) CHIP carriers, and carriers of specific CHIP gene mutations, all samples were used (i.e. no exclusions based on cancer diagnoses). As a sensitivity analysis, all analyses were then repeated after excluding samples that had a diagnosis of any malignant cancer prior to sample collection date (n = 40, 912). Cancer phenotypes definitions were derived from medical records indicating the following ICD10 codes: C81-C96, D46, D47.1, D47.3, D47.4 for blood cancers, C81-C86, C91 for lymphoid cancers, C90, C92, C94.4, C94.6, D45, D46, D47.1, D47.3, D47.4 for myeloid cancers, C50 for breast cancers, C34 for lung cancers, C61 for prostate cancers, and C78 for colon cancers. For blood cancers, cases were also included that self-reported having leukemia, lymphoma, or multiple myeloma. These models were implemented with the same covariates and in the same fashion as described above, although models estimating risk for sex specific cancers (i.e., prostate and breast) restricted to individuals of the relevant sex and did not adjust for sex as a covariate. For smoking stratified modeling of blood and lung cancer, a stricter definition of smoking (ever vs never) was used, and included pack years as a covariate in models testing risk among smokers.

### Genetic Association Analyses of CHIP Mutation Carrier Status

Genetic association analyses of CHIP mutation carrier status in the UKB cohort to identify germline loci associated with the risk of developing CHIP was first conducted. In the common variant (MAF > 0.5%) GWAS, which included 25,657 cases and 342,866 controls of European ancestry, 27 loci were identified, including 24 novel loci and 57 independent signals (data not shown). To confirm these signals, a replication analysis in 9,523 CHIP cases and 105,502 controls of European ancestry from the GHS cohort was conducted. Of the 57 independent signals, 53 had directionally consistent effects, 14 of the 27 sentinel variants at the associated loci were nominally significant (p < 0.05), and 5 were significant at a Bonferroni level of significance (P < 0.0019).

Since the CHIP phenotype we constructed is based on the presence of rare variants in recurrently mutated genes, rare variant and gene burden associations from genome-wide analysis will feature strong and artifactual associations with the same variants and genes through which the phenotype is defined. Furthermore, other associated rare variants (i.e., those which were not used to condition the CHIP phenotypes) may themselves be somatic variants, which achieve higher VAFs during hematopoietic clonal expansion and become indistinguishable from germline variants by best practice variant calling. While associations with variants used to define our phenotypes can serve as positive controls, this circularity was addressed by filtering out all such variants from the exemplified results. To address the potential that other variant associations are driven by somatic variants, whether significantly associated rare variants were assessed, as well as variants making up significantly associated gene burden aggregation signals, had low variant allele fractions (VAFs) across carriers, as well as whether an individual’s age at sample collection was associated with being a carrier of these variants (as this would suggest somaticism). For genome-wide significant rare variant and gene burden associations for which there was exome data, these VAF and age-association results are reported along with genetic association results and used to provide resolution as to whether such associations are likely to be driven by germline or somatic variation.

### Individual CHIP Gene Mutation Carrier Association Analyses

Among loci associated with multiple CHIP subtypes, genome-wide significant association signals were observed at the TCL1A locus that were not present in the overall CHIP analysis. This locus is notable because it exhibited opposing effects across CHIP subtypes (data not shown), with lead SNPs (e.g. rs2887399-T, rs11846938-G) at the locus associated with an increased risk of DNMT3A-CHIP (OR = 1.14, P = 1.8 * 10⁻²⁰), and a reduced risk of TET2-CHIP (OR = 0.77, P = 2.3 * 10⁻²¹) and ASXL1-CHIP (OR = 0.66, P = 4.8 * 10⁻¹⁸). Effect estimates from the other five CHIP gene specific association analyses are also consistent with protective effects (data not shown). This suggests that DNMT3A-CHIP is unique among clonal hematopoietic subtypes with regard to the genetic influence of the TCL1A locus.

### Genetic Comparisons Between CHIP, mCA, mLOY, mLOX, and Telomere Length

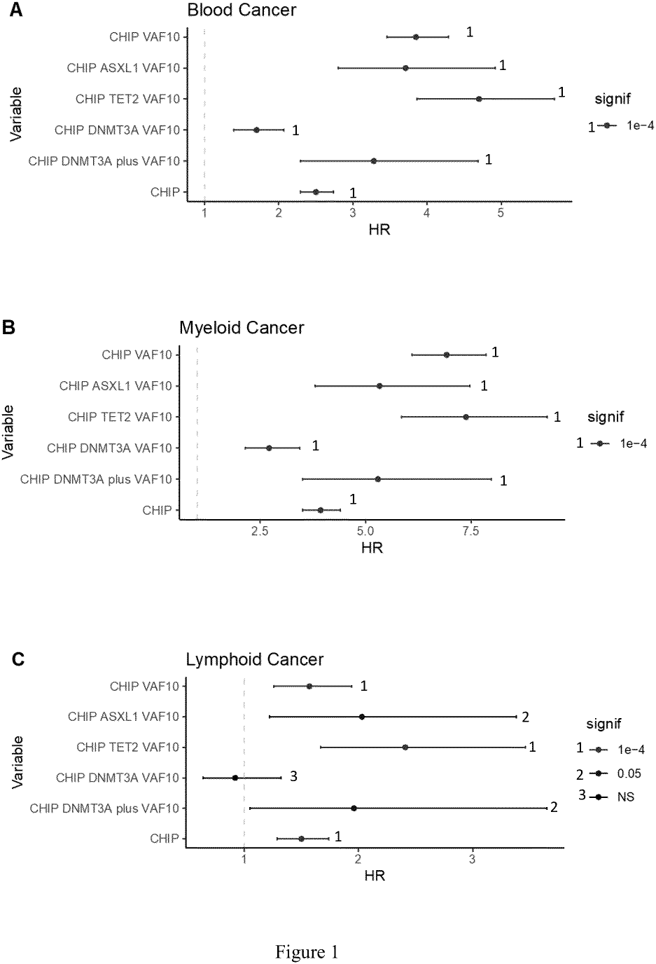

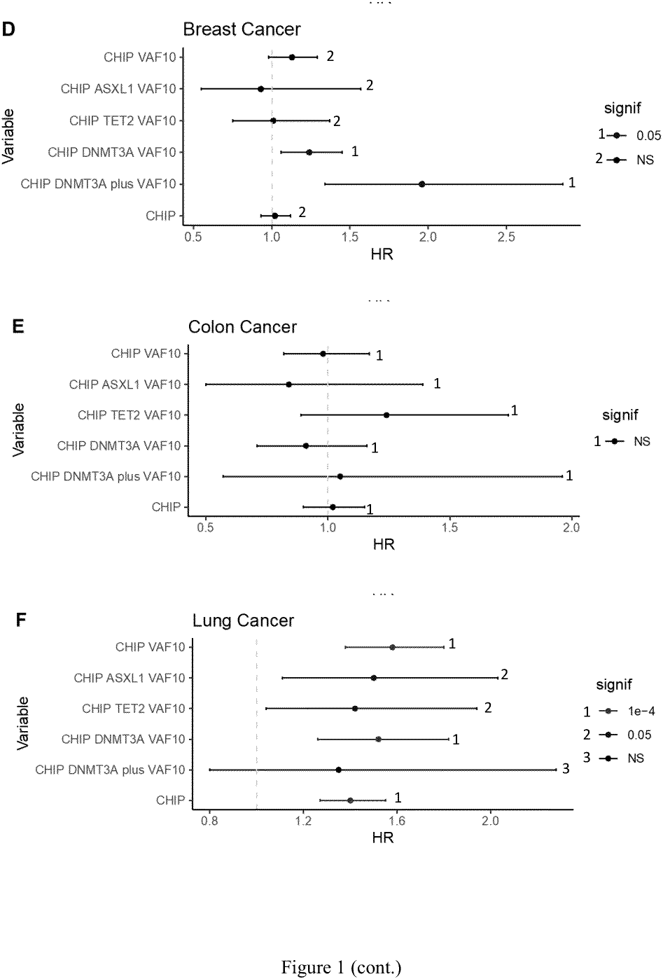

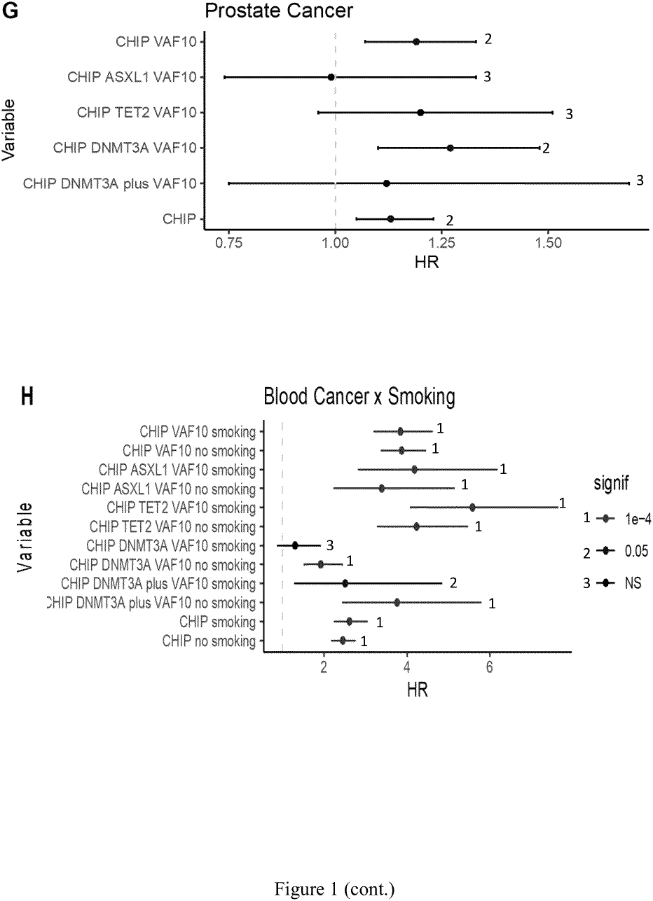

To evaluate the relationship between CHIP and other forms of somatic alterations of the blood, phenotype information on other types of clonal hematopoiesis and on telomere length that are available on UKB participants was utilized (Zekavat et al., Nature Med., 2021, 27, 1012-1024; Thompson et al., Nature, 2019, 575, 652-657; Loh et al., Nature, 2018, 559, 350-355; and Codd et al, Polygenic basis and biomedical consequences of telomere length variation, 2021, medRxiv 2021.03.23.21253516, doi:10.1101/2021.03.23.21253516). The phenotypic overlap between CHIP and mLOX, mLOY, and autosomal mosaic chromosomal alterations (mCAaut) was first evaluated. CHIP is distinct from mCA phenotypes (mCAaut, mLOX, and mLOY), with >80% of CHIP carriers having no identified mCAs (data not shown). Carriers of only a single CH phenotype (i.e. CHIP, mLOY, mLOX, or mCAaut) were younger on average than those with multiple CH lesions, and mCAaut and CHIP carriers were youngest among single CH phenotype carriers (FIG. 1, Panel B). The fact that mLOY occurs in older individuals but is also relatively common suggests that the processes driving and/or following such clonal genetic loss happen more quickly than do other somatic alterations.

### Longitudinal Analysis of Hematological and Oncological Risk Within Individuals With CHIP

Given the confounding that can bias cross-sectional association analyses, survival analyses was performed to evaluate whether individuals with CHIP at the time of enrollment in UKB were at an elevated risk of incident cardiovascular disease, cancer, and all-cause mortality. To do this, aggregate longitudinal phenotypes of lymphoid cancer, myeloid cancer, lung cancer, breast cancer, prostate cancer, colon cancer, and overall survival (i.e. Any Death) were generated. Because prior longitudinal studies of CHIP and the risk of many of these outcomes have focused on high VAF CHIP, we focused on CHIP carriers with VAF >= 0.10 for these analyses.

Whether CHIP carriers are at an increased risk of hematologic and solid cancers, was next tested and whether risk differed by CHIP mutational subtype for the three most common CHIP genes (i.e., DNMT3A, TET2, ASXL1, Table S40). To control for the possibility that toxic chemotherapeutic treatment for previous cancers might drive the development of CHIP mutations (Smith et al., J. Nat′l Cancer Inst., 1996, 88, 407-418), and/or otherwise confound association analyses, all analyses were repeated after excluding individuals with any diagnoses of cancer prior to DNA collection. As expected, CHIP carriers were found with VAF >= 0.10 to be at a significantly elevated risk of developing any blood cancer (HR = 3.85 [3.46-4.29], P = 3.70 * 10⁻¹³¹). TET2 mutation carriers were at the greatest risk of developing blood cancers (HR = 4.70 [3.86-5.72], P = 1.50 * 10⁻⁵³), whereas DNMT3A mutation carriers had much more modest risk of acquiring blood cancers (HR = 1.70 [1.39-2.07], P = 3.00 * 10⁻⁷) unless they also had at least one additional CHIP mutation (HR = 3.28 [2.29-4.69], P = 9.90 * 10⁻¹¹; FIG. 1, Panel A). When decomposing blood cancers into myeloid and lymphoid subtypes, it was estimated that high VAF CHIP carriers were at a significantly elevated risk of developing myeloid cancers (HR = 6.92 [6.10-7.86], P = 1.20 * 10⁻¹⁹⁵, FIG. 1, Panel B) compared with lymphoid cancers (HR = 1.57 [1.26-1.94], P = 3.90 * 10⁻⁵, FIG. 1, Panel C). Furthermore, it was estimated that DNMT3A mutations do not predispose to lymphoid carriers (HR = 0.92 [0.64-1.32], P = 0.66, FIG. 1, Panel 7C). We then tested whether CHIP carriers were at an elevated risk of developing solid tumors (FIG. 1, Panels D, E, F, and G), and found that high VAF carriers are at significantly elevated risk of developing lung cancer (HR = 1.58 [1.38-1.80], P = 2.90 * 10⁻¹¹), modestly increased risk of developing prostate cancer (HR = 1.19 [1.07-1.33], P = 1.90 * 10⁻³), and nominally increased risk of developing breast cancers (HR = 1.13 [0.98-1.29], P = 0.082). No increased risk for the development of colon cancer (HR = 0.98 [0.82-1.17], P = 0.84) was found. Models estimating event risk on the basis of CHIP mutational subtype (e.g. carriers must have DNMT3A mutations) suggest that these associations with prostate and breast cancer are driven primarily by DNMT3A mutations (FIG. 1).

Given the strong associations between CHIP and blood cancer and lung cancer, and the associations between smoking and both CHIP and lung cancer, additional analyses were performed stratified by smoking status to test whether these associations were driven by smoking and merely marked by CHIP mutations (data not shown). High VAF CHIP carriers are at an elevated risk of developing blood cancers in smokers (HR = 3.84 [3.21-4.60], P = 1.30 * 10⁻¹¹) and non-smokers (HR = 3.87 [3.38-4.44], P = 5.70 * 10⁻⁸⁵), and while this pattern was similar among carriers of TET2 and ASXL1 CHIP subtypes, DNMT3A carriers were only at a significantly increased risk of blood cancer in non-smokers (FIG. 1, Panel H). Notably, lung cancer risk for high VAF CHIP carriers (compared with healthy controls) is significantly elevated among both smokers and non-smokers, and is in fact higher in non-smokers (HRₙₒₙ₋ₛₘₒₖₑᵣₛ = 1.83 [1.41-2.38], P = 5.80 * 10⁻⁶, HRₛₘₒₖₑᵣₛ = 1.56 [1.33-1.82], P = 2.50 * 10^{-8,}FIG. 1, Panel I). These associations are driven by DNMT3A and ASXL1 CHIP carriers, with both estimated to have elevated lung cancer risk in both smokers and non-smokers (FIG. 1, Panel I). Overall, these models suggest that CHIP mutation carriers are at an elevated risk of blood cancer and lung cancer independent of smoking, but that CHIP is likely also marking additional blood cancer risk that results from smoking.

### Single Cell Transcriptomic Analysis Supports the Expression of CH Associated Genes in Hematopoietic Stem and Progenitor Cells

Because CHIP was associated with variation in a number of hematological binary and quantitative traits, it was investigated where genes in CHIP-associated loci might exert their effects in the blood compartment. To do this, a publicly available single-cell RNA sequencing data set of bone marrow mononuclear cells was leveraged (Stuart et al., Cell, 2019, 177, 1888-1902; and Hao et al., Cell, 2021). A gene list of interest was assembled by choosing the nearest gene to every common index SNP and rare variant that was significantly associated with any CHIP or CH phenotype, as well as genes that were significantly associated with any phenotype in our gene burden testing analyses. This left with 258 unique genes of interest, which was then used to query expression patterns across cell types from the hematopoietic compartment.

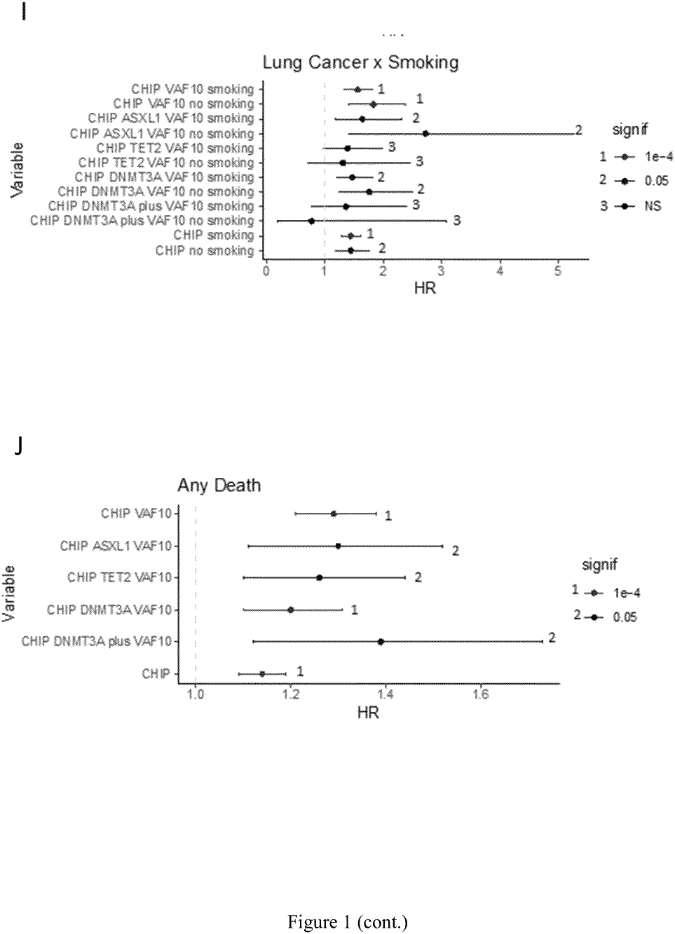

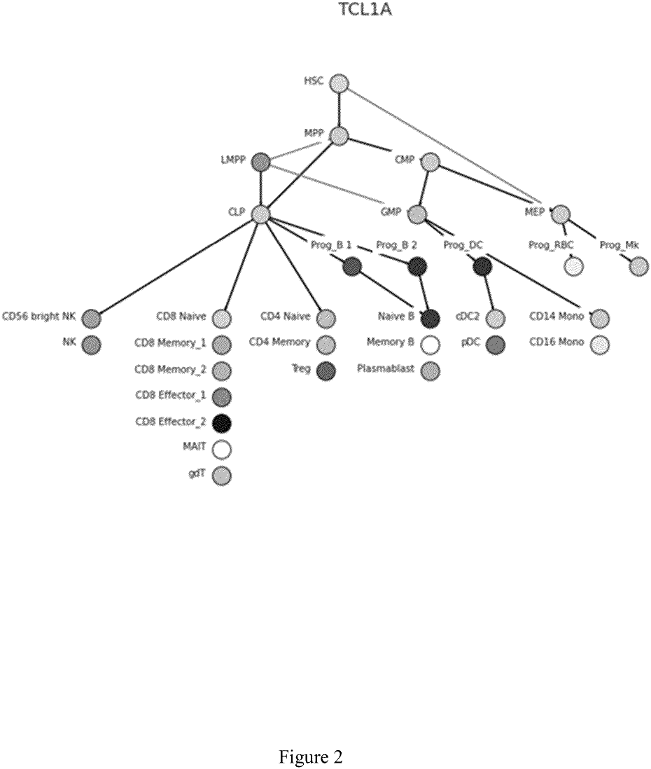

Using the Gini coefficient as a measure of cell type specificity, it was tested whether any genes were significantly enriched in any cell types. While no genes were significantly enriched after multiple testing correction, the most cell-specific gene was TCL1A (FIG. 2), which was strongly expressed in B cells.

### Discussion

Perhaps most unexpectedly, DNMT3A-CHIP was not at all associated with incident myeloid leukemia. The fact that significant associations were found between myeloid leukemia and DNMT3A mutation carriers that also have other CHIP mutations, but not individuals conditioned only on the presence of DNMT3A mutations (i.e., agnostic to whether they have other CHIP mutations), helped to isolate the effects on disease risk of DNMT3A vs other CHIP mutations. This pattern was pronounced for blood cancer overall, and myeloid cancer in particular, and suggested that hematologic malignancies were predominantly driven by non-DNMT3A mutations (and by TET2 specifically, among the most recurrently mutated CHIP genes). This pattern was not seen across CHIP associations with solid tumors, which was estimated to be driven predominantly by DNMT3A. Interestingly, in UK Biobank, out of the 3,484 individuals excluded due to their diagnosis of blood cancer prior to DNA collection and sequencing, 272 (7.8%) had DNMT3A mutations, and out of the 40,912 individuals excluded due to their diagnosis of any cancer prior to DNA collection and sequencing, 2,103 (5.1%) had DNMT3A mutations. 18.75% of the DNMT3A carriers with blood cancer prior to sequencing had CHIP mutations in multiple genes, whereas only 6.6% of the DNMT3A carriers with any cancer prior to sequencing had CHIP mutations in multiple genes. Therefore, the inclusion of such individuals in the analyses done by prior studies, and/or the failure to identify DNMT3A carriers as having other CHIP mutations as well, may have led to the misestimation of DNMT3A specific risk. On the whole, these results further clarified the role of CHIP mutational subtypes in the development of cancer and emphasize the importance of viewing (and potentially treating) different CHIP subtypes as distinct hematologic pre-conditions.

### Example 2: Treatment of Subjects Having CHIP With Donor HSCs Treated Ex Vivo with TCL1A Antagonists

HSCs can be isolated from donor adult bone marrow via apheresis or from banked human umbilical cord blood, and then exposed to one or more TCL1A antagonizing strategies (e.g. RNA knockdown, small molecule inhibitor, antisense-oligonucleotide knock-in via viral vector). After a sufficient period of time as to antagonize TCL1A driven signaling and/or stabily transfect cells, the ex vivo treated HSCs can be transplanted through a central venous catheter into a subject having CHIP or at risk of developing CHIP.

### Example 3: Treatment of Subjects Having CHIP With Autologous HSCs Treated Ex Vivo with TCL1A Antagonists

HSCs can be isolated from an individual’s own bone marrow in anticipation of autologous transplant secondary to cellular treatment and/or gene editing. Once cells are harvested via apheresis, they can be exposed to one or more TCL1A antagonizing strategies (e.g. RNA knockdown, small molecule inhibitor, antisense-oligonucleotide knock-in via viral vector). After a sufficient period of time as to antagonize TCL1A driven signaling and/or stabily transfect cells, the ex vivo treated HSCs can be transplanted through a central venous catheter into a subject having CHIP or at risk of developing CHIP.

### Example 4: Treatment of Subjects Having CHIP With Autologous HSCs Treated In Vivo with TCL1A Antagonists

Subjects having CHIP or at risk of developing CHIP that receive a hematopoietic stem cell transplant (HSCT) can be treated with one or more TCL1A antagonizing strategies (e.g. RNA knockdown, small molecule inhibitor) weeks to months after receiving the transplant. This can antagonize pathalogic CHIP clone expansion during the time while the subject’s hematopoietic system is reconsititued.

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety and for all purposes.

What is claimed is:

1. A method of treating, preventing, or reducing the development of clonal hematopoiesis of indeterminate potential (CHIP) in a subject, the method comprising administering a T Cell Leukemia/Lymphoma Protein 1A (TCL1A) antagonist to the subject, wherein the subject has a TET2-CHIP somatic mutation and/or an ASXL1-CHIP somatic mutation.

2-4. (canceled)

5. The method according to claim 1, wherein the TCL1A antagonist comprises an inhibitory nucleic acid molecule that hybridizes to a TCL1A nucleic acid molecule, wherein the inhibitory nucleic acid molecule comprises an antisense nucleic acid molecule, a small interfering RNA (siRNA), or a short hairpin RNA (shRNA).

6-11. (canceled)

12. The method according to claim 1, further comprising detecting the presence or absence of a TCL1A variant nucleic acid molecule in a biological sample from the subject.

13. The method according to claim 12, further comprising administering a therapeutic agent that treats, prevents, or reduces development of CHIP in a standard dosage amount to a subject wherein a TCL1A variant nucleic acid molecule is absent from the biological sample.

14. The method according to claim 12, further comprising administering a therapeutic agent that treats, prevents, or reduces development of CHIP in a standard dosage amount to a subject wherein a TCL1A variant nucleic acid molecule is present in the biological sample.

15. The method according to claim 12, further comprising administering a therapeutic agent that treats, prevents, or reduces development of CHIP in a dosage amount that is the same as, greater than, or less than a standard dosage amount to a subject that is heterozygous for a TCL1A variant nucleic acid molecule.

16. The method according to claim 12, further comprising administering a therapeutic agent that treats, prevents, or reduces development of CHIP in a dosage amount that is the same as, less than, or greater than a standard dosage amount to a subject that is heterozygous for a TCL1A variant nucleic acid molecule.

17. The method according to claim 12, wherein the TCL1A variant nucleic acid molecule is a missense variant, splice-site variant, a stop-gain variant, a start-loss variant, a stop-loss variant, a frameshift variant, or an in-frame indel variant, or a variant that encodes a truncated predicted loss-of-function polypeptide.

18. The method according to claim 12, wherein the TCL1A variant nucleic acid molecule comprises the rs2296311, rs2887399, or rs11846938 single nucleotide polymorphism.

19. A method of treating a subject with a therapeutic agent that treats, prevents, or reduces development of CHIP, wherein the subject has CHIP or is at risk of developing CHIP, and wherein the subject comprises a TET2-CHIP mutation, the method comprising:
determining whether the subject has a TCL1A variant nucleic acid molecule by:
obtaining or having obtained a biological sample from the subject; and
performing or having performed a sequence analysis on the biological sample to determine if the subject has a genotype comprising TCL1A variant nucleic acid molecule; and
administering or continuing to administer the therapeutic agent that treats, prevents, or reduces development of CHIP in a standard dosage amount to a subject that is TCL1A reference; and/or administering a TCL1A antagonist to the subject;
administering or continuing to administer the therapeutic agent that treats, prevents, or reduces development of CHIP in an amount that is the same as, greater than, or less than a standard dosage amount to a subject that is heterozygous for the TCL1A variant nucleic acid molecule; and/or administering a TCL1A antagonist to the subject; or
administering or continuing to administer the therapeutic agent that treats, prevents, or reduces development of CHIP in an amount that is the same as, greater than, or less than a standard dosage amount to a subject that is homozygous for the TCL1A variant nucleic acid molecule;
wherein the presence of a genotype having the TCL1A variant nucleic acid molecule indicates the subject has a decreased risk of developing CHIP.

20. (canceled)

21. The method according to claim 19, wherein the subject is TCL1A reference, and the subject is administered or continued to be administered the therapeutic agent that treats, prevents, or reduces development of CHIP in a standard dosage amount, and is administered the TCL1A antagonist.

22. The method according to claim 19, wherein the subject is heterozygous for the TCL1A variant nucleic acid molecule, and the subject is administered or continued to be administered the therapeutic agent that treats, prevents, or reduces development of CHIP in an amount that is the same as, greater than, or less than a standard dosage amount, and is administered the TCL1A antagonist.

23. The method according to claim 19, wherein the TCL1A variant nucleic acid molecule is a missense variant, splice-site variant, a stop-gain variant, a start-loss variant, a stop-loss variant, a frameshift variant, or an in-frame indel variant, or a variant that encodes a truncated predicted loss-of-function polypeptide.

24. The method according to claim 19, wherein the TCL1A variant nucleic acid molecule comprises the rs2296311, rs2887399, or rs11846938 single nucleotide polymorphism.

25. The method according to claim 19, wherein the TCL1A antagonist comprises an inhibitory nucleic acid molecule that hybridizes to a TCL1A nucleic acid molecule, wherein the inhibitory nucleic acid molecule comprises an antisense nucleic acid molecule, a small interfering RNA (siRNA), or a short hairpin RNA (shRNA).

26-31. (canceled)

32. A method of treating a subject with a therapeutic agent that treats, prevents, or reduces development of CHIP, wherein the subject has CHIP or is at risk of developing CHIP, and wherein the subject comprises an ASXL1-CHIP mutation, the method comprising:
determining whether the subject has a TCL1A variant nucleic acid molecule by:
obtaining or having obtained a biological sample from the subject; and
performing or having performed a sequence analysis on the biological sample to determine if the subject has a genotype comprising TCL1A variant nucleic acid molecule; and
administering or continuing to administer the therapeutic agent that treats, prevents, or reduces development of CHIP in a standard dosage amount to a subject that is TCL1A reference; and/or administering a TCL1A antagonist to the subject;
administering or continuing to administer the therapeutic agent that treats, prevents, or reduces development of CHIP in an amount that is the same as, greater than, or less than a standard dosage amount to a subject that is heterozygous for the TCL1A variant nucleic acid molecule; and/or administering a TCL1A antagonist to the subject; or
administering or continuing to administer the therapeutic agent that treats, prevents, or reduces development of CHIP in an amount that is the same as, greater than, or less than a standard dosage amount to a subject that is homozygous for the TCL1A variant nucleic acid molecule;
wherein the presence of a genotype having the TCL1A variant nucleic acid molecule indicates the subject has a decreased risk of developing CHIP.

33. (canceled)

34. The method according to claim 32, wherein the subject is TCL1A reference, and the subject is administered or continued to be administered the therapeutic agent that treats, prevents, or reduces development of CHIP in a standard dosage amount, and is administered the TCL1A antagonist.

35. The method according to claim 32, wherein the subject is heterozygous for the TCL1A variant nucleic acid molecule, and the subject is administered or continued to be administered the therapeutic agent that treats, prevents, or reduces development of CHIP in an amount that is the same as, greater than, or less than a standard dosage amount, and is administered the TCL1A antagonist.

36. The method according to claim 32, wherein the TCL1A variant nucleic acid molecule is a missense variant, splice-site variant, a stop-gain variant, a start-loss variant, a stop-loss variant, a frameshift variant, or an in-frame indel variant, or a variant that encodes a truncated predicted loss-of-function polypeptide.

37. The method according to claim 32, wherein the TCL1A variant nucleic acid molecule comprises the rs2296311, rs2887399, or rs11846938 single nucleotide polymorphism.

38. The method according to claim 32, wherein the TCL1A antagonist comprises an inhibitory nucleic acid molecule that hybridizes to a TCL1A nucleic acid molecule, wherein the inhibitory nucleic acid molecule comprises an antisense nucleic acid molecule, a small interfering RNA (siRNA), or a short hairpin RNA (shRNA).

39-83. (canceled)


SEQUENCE LISTING (4 sequences)

SEQ ID NO: 1 - DNA, 5230 bp, Homo sapiens
  source 1..5230: mol_type = genomic DNA
tcctttgccaaaaaacgtactctgacactcaaaggcacaggctggtggagatccagggaacccgggaaagcccaggcctaactcaggcccgcgcgggtctcttctgatcccataagatgaggagtgggtcctggctctgggcatcctggtgtcgactgtgagttcccagcagaggcccagagtcccggtccggcagccgagggaagcgggggggtcttccagaagaagaaagggccaaggtcaccccggtgcctctccagcagcagcagagggcggcggtcggtgtcgctgctggccggggcctcgaggaaggcgcgggccagctggggccgggtctgcgttcccaggagctgccaccgttccagggagcaagtcaggccgggacgtagcgcctgcgcgggaccctcacttgccaccaagacccccacaaaccccgccccatcctgccttacgccccgccccaaggtcgttctcccgacccggggtcccgccccaaggccgtcctcccgccccgccgcttggtggcggccgcatgctgcccggatataaagggtcggccccacatcccagggaccagcgagcggccttgagaggctctggctcttgcttcttaggcggcccgaggacgccatggccgagtgcccgacactcggggaggcagtcaccgaccacccggaccgcctgtgggcctgggagaagttcgtgtatttggacgagaagcagcacgcctggctgcccttaaccatcgaggtacagccagctttggagcggatggcgaggcagcaggggcagcccctgggagcttgggatgccctaggaagggcgaggactcaaggagcactcactatggggcagggaggatccccacagatgaagccactttgagccgggctctggaggatgaataggagttctccaggcagggaagaagggtgggaaaaccgcaaaggaatgtcggtcaaaggggtggacccagtgcctgtggagtgtgactataatgttgactacagcaggcattttctgggcttcggggtcctaatccttaaaaatgggtatctctaagtgactcatccatatggccgattatcggaatcatctcaggtgggtcccagaaatctgtatttttaaaaagaaccctcaacagtttagggtcaaccaggcatagcaaacactggcctagagtgtgaagtattttccactacctctgggctttatttagaaaacaaatttaacaagtgatgtcgtagtataagcgccggtattgaatcaatattgacttttttaatgtgtgatgccttaagatgggtccttaatccatgttaagtttttgttaaagaaatagataagtcttttacaagtatttggatttactcaatgaaaaagagtcaaaaaatgttcaaactctctccaaacatacactgaagaaagcataaaaattaaaaaatatattagaacacgtatgtccagtagcaatcaaaaattattgagtgttgactgtgtctctacagatgggaaactgaggcacaaaaatgtacatttgtccgaggtaagattgctagtaggtaatggggttggaattctaggctcttaaccaccacaaaatctgcatttttattggcatttcaattttttaaatatgtttttactttaaaaatcaagttaaatacttacttttttaaaatcaaaatttgaagaaataatttgaagattcagtggatttctttttttaaatctctgagaaatctcttcttcaacgtgacaccaaaaccacgaaccagacagtttttcataaaatcatgatacatgctccacaaaaaataacccactagcaaactgtgggacagattttgcctcacatcattgaaaaggccagtattctttttctctctttctttctttgttgttttttttttttcctgtagatacagggtctcgctctgtgacccaggctggtcttaaactcctggcctcaagcgatcctcctgcctctgccttccaaagcactggaattacaagtgtgagccgctgcaacccgccagaaaaaagtgtgcctttcatggccctgtctgggtggctagacacgtgtgtgtgctggtggtcctggcccagccagagtccctgagaggagcatgcatggcctaaggaagtgagcttcagggaacagtgatgaccatcatttcacactcggacccctgcccagatgggtggatggctggcaggagggattccggttttctgcgcctggagaagccctggcaagcggaacctgaaagtatgccctgtccttttcttctcctcagataaaggataggttacagttacgggtgctcttgcgtcgggaagacgtcgtcctggggaggcctatgacccccacccagataggcccaagcctgctgcctatcatgtggcagctctaccctgatggacgataccgatcctcagactccagtttctggcgcttagtgtaccacatcaaggtgagtgtctttctcccagaggtccatcgggtgatcttgggtttgccctcctccatgtctggccttagtggtttatcttccctccatcccagtcccaaaagcattaaaaaatgggggaaatgtacaagtgcagatttctaaaggactttaccagagagaagatagatcctctgaggtctctaacagaaccctacctccacttcctcccagccaccagctagccgcaggaagacatctctggtgggcagtcacaggctgaaggctggtgggaggaggggcagtctccaagagcccctgaaatcctcacacctgggttcctacctgctgtttccagctaggggaaggcgcaggagtgaggaatggagggagtggagggctctggccgatcaatgccttctctctctctctgcctctcagattgacggcgtggaggacatgcttctcgagctgctgccagatgactgatgtatggtgagctccactggagcctgacccctcttagtccacagtggctgtatcagaaagaaagaccaccccttctccatgaaggcagtgctaacccctccccgactgctgccatctgagggtccctagggatgggagaggcttcctggaggcactcatgtctcccttaccacttcgggagccaagggctttgtaggcagccccctttatcgcagctgctcatatctataaagtacttcacaagtttcagctggcactttcatttaccattgcttttttttttttttgagatgagtctggctctgtggcccaggctagagtgtagtggtgcaatctcagctcactgcaagctctgcctcccgggttcacaccattctcctgcctcagccctcggagtagctgggactacaggcgcccgccaccacacctggctaattttttttttttttttttttttttagtagagacggggtttcaccgtgttagccaggatggtctcgatctcctgacctcatgatctgcccgcctcggcctcccaaagtgctgggattacaggcatgagccaccacgtccggccttaccattgctttattaaataagcactggtgcttgattatatcagctgagccagatattagatacgctattgagttttgaggaaataagagtacaaaactcagaaatgagttgaagtatagtgacatcttcagattacagacccaggtgtcagaatttgccttggctcagaaggcctctgggggccatccctgaccactaggcttcccacttagacctgctccagcagcaccacccctcgccactgcctggtcctttccttcacccttgattctgtcttcttttgtccttctccaggtcttggcagcacctgtctcctttcaccccagggcctgagcctggccagcctacaatggggatgttgtgtttctgttcaccttcgtttactatgcctgtgtcttctccaccacgctggggtctgggaggaatggacagacagaggatgagctctacccagggcctgcaggacctgcctgtagcccactctgctcgccttagcactaccactcctgccaaggaggattccatttggcagagcttcttccaggtgcccagctatacctgtgcctcggcttttctcagctggatgatggtcttcagcctctttctgtcccttctgtccctcacagcactagtatttcatgttgcacacccactcagctccgtgaacttgtgagaacacagccgattcacctgagcaggacctctgaaaccctggaccagtggtctcacatggtgctacgcctgcatgtaaacacgcctgcaaacgctgcctgccggtaaacacgcctgcaaacgctgcctgcccgtaaacacgcctgcaaacgctgcctgcccacacaggttcacgtgcagctcaaggaaaggcctgaaaggagcccttatctgtgctcaggactcagaagcctctgggtcagtggtccacatcccgggacgcagcaggaggccaggccggcgagccctgtggatgagccctcagaacccttggcttgcccacgtggaaaagggatagaggttgggtttccccccttttatagatggtcacgcacctgggtgttacaaagttgtatgtggcatgaatactttttgtaatgattgattaaatgcaagatagtttatctaacttcgtgcggaatcagcttctatccttgacttagattctggtggagagaagtgagaataggcagcccccaaataaaaaatattcatggatttatttattttcctttggagataagggactacaattttttccccgtgtaatccaatgcttaaaaccccagtgttcttggaggcatacgttgagaaccattggagtaagcaacctcaaaaatgttgttggcaatgcccggtattccagagccccccctgcctccatctggtcctgccctccttaccctcctcattcccatgtgccctcggccccagccagctctgcctcctccttacctgggcattcaccaggctctgtccttgcacagataacttctatcctctctgctcacaatcccagcagaagcccctcctgcctgaagctgtccctgaccactcaggccccaagcagactcccttccccacctcccatgacctgcagtcacagcagatcccaccctgtgccctgtcctcttgctcccttctcaagaccctgctgcccctcctgtacctctatagacagtagctggacctcagatattaccaggggatgccttcca

SEQ ID NO: 2 - DNA, 5230 bp, Homo sapiens
  source 1..5230: mol_type = genomic DNA
tcctttgccaaaaaacgtactctgacactcaaaggcacaggctggtggagatccagggaacccgggaaagcccaggcctaactcaggcccgcgcgggtctcttctgatcccataagatgaggagtgggtcctggctctgggcatcctggtgtcgactgtgagttcccagcagaggcccagagtcccggtccggcagccgagggaagcgggggggtcttccagaagaagaaagggccaaggtcaccccggtgcctctccagcagcagcagagggcggcggtcggtgtcgctgctggccggggcctcgaggaaggcgcgggccagctggggccgggtctgagttcccaggagctgccaccgttccagggagcaagtcaggccgggacgtagcgcctgcgcgggaccctcacttgccaccaagacccccacaaaccccgccccatcctgccttacgccccgccccaaggtcgttctcccgacccggggtcccgccccaaggccgtcctcccgccccgccgcttggtggcggccgcatgctgcccggatataaagggtcggccccacatcccagggaccagcgagcggccttgagaggctctggctcttgcttcttaggcggcccgaggacgccatggccgagtgcccgacactcggggaggcagtcaccgaccacccggaccgcctgtgggcctgggagaagttcgtgtatttggacgagaagcagcacgcctggctgcccttaaccatcgaggtacagccagctttggagcggatggcgaggcagcaggggcagcccctgggagcttgggatgccctaggaagggcgaggactcaaggagcactcactatggggcagggaggatccccacagatgaagccactttgagccgggctctggaggatgaataggagttctccaggcagggaagaagggtgggaaaaccgcaaaggaatgtcggtcaaaggggtggacccagtgcctgtggagtgtgactataatgttgactacagcaggcattttctgggcttcggggtcctaatccttaaaaatgggtatctctaagtgactcatccatatggccgattatcggaatcatctcaggtgggtcccagaaatctgtatttttaaaaagaaccctcaacagtttagggtcaaccaggcatagcaaacactggcctagagtgtgaagtattttccactacctctgggctttatttagaaaacaaatttaacaagtgatgtcgtagtataagcgccggtattgaatcaatattgacttttttaatgtgtgatgccttaagatgggtccttaatccatgttaagtttttgttaaagaaatagataagtcttttacaagtatttggatttactcaatgaaaaagagtcaaaaaatgttcaaactctctccaaacatacactgaagaaagcataaaaattaaaaaatatattagaacacgtatgtccagtagcaatcaaaaattattgagtgttgactgtgtctctacagatgggaaactgaggcacaaaaatgtacatttgtccgaggtaagattgctagtaggtaatggggttggaattctaggctcttaaccaccacaaaatctgcatttttattggcatttcaattttttaaatatgtttttactttaaaaatcaagttaaatacttacttttttaaaatcaaaatttgaagaaataatttgaagattcagtggatttctttttttaaatctctgagaaatctcttcttcaacgtgacaccaaaaccacgaaccagacagtttttcataaaatcatgatacatgctccacaaaaaataacccactagcaaactgtgggacagattttgcctcacatcattgaaaaggccagtattctttttctctctttctttctttgttgttttttttttttcctgtagatacagggtctcgctctgtgacccaggctggtcttaaactcctggcctcaagcgatcctcctgcctctgccttccaaagcactggaattacaagtgtgagccgctgcaacccgccagaaaaaagtgtgcctttcatggccctgtctgggtggctagacacgtgtgtgtgctggtggtcctggcccagccagagtccctgagaggagcatgcatggcctaaggaagtgagcttcagggaacagtgatgaccatcatttcacactcggacccctgcccagatgggtggatggctggcaggagggattccggttttctgcgcctggagaagccctggcaagcggaacctgaaagtatgccctgtccttttcttctcctcagataaaggataggttacagttacgggtgctcttgcgtcgggaagacgtcgtcctggggaggcctatgacccccacccagataggcccaagcctgctgcctatcatgtggcagctctaccctgatggacgataccgatcctcagactccagtttctggcgcttagtgtaccacatcaaggtgagtgtctttctcccagaggtccatcgggtgatcttgggtttgccctcctccatgtctggccttagtggtttatcttccctccatcccagtcccaaaagcattaaaaaatgggggaaatgtacaagtgcagatttctaaaggactttaccagagagaagatagatcctctgaggtctctaacagaaccctacctccacttcctcccagccaccagctagccgcaggaagacatctctggtgggcagtcacaggctgaaggctggtgggaggaggggcagtctccaagagcccctgaaatcctcacacctgggttcctacctgctgtttccagctaggggaaggcgcaggagtgaggaatggagggagtggagggctctggccgatcaatgccttctctctctctctgcctctcagattgacggcgtggaggacatgcttctcgagctgctgccagatgactgatgtatggtgagctccactggagcctgacccctcttagtccacagtggctgtatcagaaagaaagaccaccccttctccatgaaggcagtgctaacccctccccgactgctgccatctgagggtccctagggatgggagaggcttcctggaggcactcatgtctcccttaccacttcgggagccaagggctttgtaggcagccccctttatcgcagctgctcatatctataaagtacttcacaagtttcagctggcactttcatttaccattgcttttttttttttttgagatgagtctggctctgtggcccaggctagagtgtagtggtgcaatctcagctcactgcaagctctgcctcccgggttcacaccattctcctgcctcagccctcggagtagctgggactacaggcgcccgccaccacacctggctaattttttttttttttttttttttttagtagagacggggtttcaccgtgttagccaggatggtctcgatctcctgacctcatgatctgcccgcctcggcctcccaaagtgctgggattacaggcatgagccaccacgtccggccttaccattgctttattaaataagcactggtgcttgattatatcagctgagccagatattagatacgctattgagttttgaggaaataagagtacaaaactcagaaatgagttgaagtatagtgacatcttcagattacagacccaggtgtcagaatttgccttggctcagaaggcctctgggggccatccctgaccactaggcttcccacttagacctgctccagcagcaccacccctcgccactgcctggtcctttccttcacccttgattctgtcttcttttgtccttctccaggtcttggcagcacctgtctcctttcaccccagggcctgagcctggccagcctacaatggggatgttgtgtttctgttcaccttcgtttactatgcctgtgtcttctccaccacgctggggtctgggaggaatggacagacagaggatgagctctacccagggcctgcaggacctgcctgtagcccactctgctcgccttagcactaccactcctgccaaggaggattccatttggcagagcttcttccaggtgcccagctatacctgtgcctcggcttttctcagctggatgatggtcttcagcctctttctgtcccttctgtccctcacagcactagtatttcatgttgcacacccactcagctccgtgaacttgtgagaacacagccgattcacctgagcaggacctctgaaaccctggaccagtggtctcacatggtgctacgcctgcatgtaaacacgcctgcaaacgctgcctgccggtaaacacgcctgcaaacgctgcctgcccgtaaacacgcctgcaaacgctgcctgcccacacaggttcacgtgcagctcaaggaaaggcctgaaaggagcccttatctgtgctcaggactcagaagcctctgggtcagtggtccacatcccgggacgcagcaggaggccaggccggcgagccctgtggatgagccctcagaacccttggcttgcccacgtggaaaagggatagaggttgggtttccccccttttatagatggtcacgcacctgggtgttacaaagttgtatgtggcatgaatactttttgtaatgattgattaaatgcaagatagtttatctaacttcgtgcggaatcagcttctatccttgacttagattctggtggagagaagtgagaataggcagcccccaaataaaaaatattcatggatttatttattttcctttggagataagggactacaattttttccccgtgtaatccaatgcttaaaaccccagtgttcttggaggcatacgttgagaaccattggagtaagcaacctcaaaaatgttgttggcaatgcccggtattccagagccccccctgcctccatctggtcctgccctccttaccctcctcattcccatgtgccctcggccccagccagctctgcctcctccttacctgggcattcaccaggctctgtccttgcacagataacttctatcctctctgctcacaatcccagcagaagcccctcctgcctgaagctgtccctgaccactcaggccccaagcagactcccttccccacctcccatgacctgcagtcacagcagatcccaccctgtgccctgtcctcttgctcccttctcaagaccctgctgcccctcctgtacctctatagacagtagctggacctcagatattaccaggggatgccttcca

SEQ ID NO: 3 - DNA, 5230 bp, Homo sapiens
  source 1..5230: mol_type = genomic DNA
tcctttgccaaaaaacgtactctgacactcaaaggcacaggctggtggagatccagggaacccgggaaagcccaggcctaactcaggcccgcgcgggtctcttctgatcccataagatgaggagtgggtcctggctctgggcatcctggtgtcgactgtgagttcccagcagaggcccagagtcccggtccggcagccgagggaagcgggggggtcttccagaagaagaaagggccaaggtcaccccggtgcctctccagcagcagcagagggcggcggtcggtgtcgctgctggccggggcctcgaggaaggcgcgggccagctggggccgggtctgcgttcccaggcgctgccaccgttccagggagcaagtcaggccgggacgtagcgcctgcgcgggaccctcacttgccaccaagacccccacaaaccccgccccatcctgccttacgccccgccccaaggtcgttctcccgacccggggtcccgccccaaggccgtcctcccgccccgccgcttggtggcggccgcatgctgcccggatataaagggtcggccccacatcccagggaccagcgagcggccttgagaggctctggctcttgcttcttaggcggcccgaggacgccatggccgagtgcccgacactcggggaggcagtcaccgaccacccggaccgcctgtgggcctgggagaagttcgtgtatttggacgagaagcagcacgcctggctgcccttaaccatcgaggtacagccagctttggagcggatggcgaggcagcaggggcagcccctgggagcttgggatgccctaggaagggcgaggactcaaggagcactcactatggggcagggaggatccccacagatgaagccactttgagccgggctctggaggatgaataggagttctccaggcagggaagaagggtgggaaaaccgcaaaggaatgtcggtcaaaggggtggacccagtgcctgtggagtgtgactataatgttgactacagcaggcattttctgggcttcggggtcctaatccttaaaaatgggtatctctaagtgactcatccatatggccgattatcggaatcatctcaggtgggtcccagaaatctgtatttttaaaaagaaccctcaacagtttagggtcaaccaggcatagcaaacactggcctagagtgtgaagtattttccactacctctgggctttatttagaaaacaaatttaacaagtgatgtcgtagtataagcgccggtattgaatcaatattgacttttttaatgtgtgatgccttaagatgggtccttaatccatgttaagtttttgttaaagaaatagataagtcttttacaagtatttggatttactcaatgaaaaagagtcaaaaaatgttcaaactctctccaaacatacactgaagaaagcataaaaattaaaaaatatattagaacacgtatgtccagtagcaatcaaaaattattgagtgttgactgtgtctctacagatgggaaactgaggcacaaaaatgtacatttgtccgaggtaagattgctagtaggtaatggggttggaattctaggctcttaaccaccacaaaatctgcatttttattggcatttcaattttttaaatatgtttttactttaaaaatcaagttaaatacttacttttttaaaatcaaaatttgaagaaataatttgaagattcagtggatttctttttttaaatctctgagaaatctcttcttcaacgtgacaccaaaaccacgaaccagacagtttttcataaaatcatgatacatgctccacaaaaaataacccactagcaaactgtgggacagattttgcctcacatcattgaaaaggccagtattctttttctctctttctttctttgttgttttttttttttcctgtagatacagggtctcgctctgtgacccaggctggtcttaaactcctggcctcaagcgatcctcctgcctctgccttccaaagcactggaattacaagtgtgagccgctgcaacccgccagaaaaaagtgtgcctttcatggccctgtctgggtggctagacacgtgtgtgtgctggtggtcctggcccagccagagtccctgagaggagcatgcatggcctaaggaagtgagcttcagggaacagtgatgaccatcatttcacactcggacccctgcccagatgggtggatggctggcaggagggattccggttttctgcgcctggagaagccctggcaagcggaacctgaaagtatgccctgtccttttcttctcctcagataaaggataggttacagttacgggtgctcttgcgtcgggaagacgtcgtcctggggaggcctatgacccccacccagataggcccaagcctgctgcctatcatgtggcagctctaccctgatggacgataccgatcctcagactccagtttctggcgcttagtgtaccacatcaaggtgagtgtctttctcccagaggtccatcgggtgatcttgggtttgccctcctccatgtctggccttagtggtttatcttccctccatcccagtcccaaaagcattaaaaaatgggggaaatgtacaagtgcagatttctaaaggactttaccagagagaagatagatcctctgaggtctctaacagaaccctacctccacttcctcccagccaccagctagccgcaggaagacatctctggtgggcagtcacaggctgaaggctggtgggaggaggggcagtctccaagagcccctgaaatcctcacacctgggttcctacctgctgtttccagctaggggaaggcgcaggagtgaggaatggagggagtggagggctctggccgatcaatgccttctctctctctctgcctctcagattgacggcgtggaggacatgcttctcgagctgctgccagatgactgatgtatggtgagctccactggagcctgacccctcttagtccacagtggctgtatcagaaagaaagaccaccccttctccatgaaggcagtgctaacccctccccgactgctgccatctgagggtccctagggatgggagaggcttcctggaggcactcatgtctcccttaccacttcgggagccaagggctttgtaggcagccccctttatcgcagctgctcatatctataaagtacttcacaagtttcagctggcactttcatttaccattgcttttttttttttttgagatgagtctggctctgtggcccaggctagagtgtagtggtgcaatctcagctcactgcaagctctgcctcccgggttcacaccattctcctgcctcagccctcggagtagctgggactacaggcgcccgccaccacacctggctaattttttttttttttttttttttttagtagagacggggtttcaccgtgttagccaggatggtctcgatctcctgacctcatgatctgcccgcctcggcctcccaaagtgctgggattacaggcatgagccaccacgtccggccttaccattgctttattaaataagcactggtgcttgattatatcagctgagccagatattagatacgctattgagttttgaggaaataagagtacaaaactcagaaatgagttgaagtatagtgacatcttcagattacagacccaggtgtcagaatttgccttggctcagaaggcctctgggggccatccctgaccactaggcttcccacttagacctgctccagcagcaccacccctcgccactgcctggtcctttccttcacccttgattctgtcttcttttgtccttctccaggtcttggcagcacctgtctcctttcaccccagggcctgagcctggccagcctacaatggggatgttgtgtttctgttcaccttcgtttactatgcctgtgtcttctccaccacgctggggtctgggaggaatggacagacagaggatgagctctacccagggcctgcaggacctgcctgtagcccactctgctcgccttagcactaccactcctgccaaggaggattccatttggcagagcttcttccaggtgcccagctatacctgtgcctcggcttttctcagctggatgatggtcttcagcctctttctgtcccttctgtccctcacagcactagtatttcatgttgcacacccactcagctccgtgaacttgtgagaacacagccgattcacctgagcaggacctctgaaaccctggaccagtggtctcacatggtgctacgcctgcatgtaaacacgcctgcaaacgctgcctgccggtaaacacgcctgcaaacgctgcctgcccgtaaacacgcctgcaaacgctgcctgcccacacaggttcacgtgcagctcaaggaaaggcctgaaaggagcccttatctgtgctcaggactcagaagcctctgggtcagtggtccacatcccgggacgcagcaggaggccaggccggcgagccctgtggatgagccctcagaacccttggcttgcccacgtggaaaagggatagaggttgggtttccccccttttatagatggtcacgcacctgggtgttacaaagttgtatgtggcatgaatactttttgtaatgattgattaaatgcaagatagtttatctaacttcgtgcggaatcagcttctatccttgacttagattctggtggagagaagtgagaataggcagcccccaaataaaaaatattcatggatttatttattttcctttggagataagggactacaattttttccccgtgtaatccaatgcttaaaaccccagtgttcttggaggcatacgttgagaaccattggagtaagcaacctcaaaaatgttgttggcaatgcccggtattccagagccccccctgcctccatctggtcctgccctccttaccctcctcattcccatgtgccctcggccccagccagctctgcctcctccttacctgggcattcaccaggctctgtccttgcacagataacttctatcctctctgctcacaatcccagcagaagcccctcctgcctgaagctgtccctgaccactcaggccccaagcagactcccttccccacctcccatgacctgcagtcacagcagatcccaccctgtgccctgtcctcttgctcccttctcaagaccctgctgcccctcctgtacctctatagacagtagctggacctcagatattaccaggggatgccttcca

SEQ ID NO: 4 - DNA, 5230 bp, Homo sapiens
  source 1..5230: mol_type = genomic DNA
tcctttgccaaaaaacgtactctgacactcaaaggcacaggctggtggagatccagggaacccgggaaagcccaggcctaactcaggcccgcgcgggtctcttctgatcccataagatgaggagtgggtcctggctctgggcatcctggtgtcgactgtgagttcccagcagaggcccagagtcccggtccggcagccgagggaagcgggggggtcttccagaagaagaaagggccaaggtcaccccggtgcctctccagcagcagcagagggcggcggtcggtgtcgctgctggccggggcctcgaggaaggcgcgggccagctggggccgggtctgcgttcccaggagctgccaccgttccagggagcaagtcaggccgggacgtagcgcctgcgcgggaccctcacttgccaccaagacccccacaaaccccgccccatcctgccttacgccccgccccaaggtcgttctcccgacccggggtcccgccccaaggccgtcctcccgccccgccgcttggtggcggccgcatgctgcccggatataaagggtcggccccacatcccagggaccagcgagcggccttgagaggctctggctcttgcttcttaggcggcccgaggacgccatggccgagtgcccgacactcggggaggcagtcaccgaccacccggaccgcctgtgggcctgggagaagttcgtgtatttggacgagaagcagcacgcctggctgcccttaaccatcgaggtacagccagctttggagcggatggcgaggcagcaggggcagcccctgggagcttgggatgccctaggaagggcgaggactcaaggagcactcactatggggcagggaggatccccacagatgaagccactttgagccgggctctggaggatgaataggagttctccaggcagggaagaagggtgggaaaaccgcaaaggaatgtcggtcaaaggggtggacccagtgcctgtggagtgtgactataatgttgactacagcaggcattttctgggcttcggggtcctaatccttaaaaatgggtatctctaagtgactcatccatatggccgattatcggaatcatctcaggtgggtcccagaaatctgtatttttaaaaagaaccctcaacagtttagggtcaaccaggcatagcaaacactggcctagagtgtgaagtattttccactacctctgggctttatttagaaaacaaatttaacaagtgatgtcgtagtataagcgccggtattgaatcaatattgacttttttaatgtgtgatgccttaagatgggtccttaatccatgttaagtttttgttaaagaaatagataagtcttttacaagtatttggatttactcaatgaaaaagagtcaaaaaatgttcaaactctctccaaacatacactgaagaaagcataaaaattaaaaaatatattagaacacgtatgtccagtagcaatcaaaaattattgagtgttgactgtgtctctacagatgggaaactgaggcacaaaaatgtacatttgtccgaggtaagattgctagtaggtaatggggttggaattctaggctcttaaccaccacaaaatctgcatttttattggcatttcaattttttaaatatgtttttactttaaaaatcaagttaaatacttacttttttaaaatcaaaatttgaagaaataatttgaagattcagtggatttctttttttaaatctctgagaaatctcttcttcaacgtgacaccaaaaccacgaaccagacagtttttcataaaatcatgatacatgctccacaaaaaataacccactagcaaactgtgggacagattttgcctcacatcattgaaaaggccagtattctttttctctctttctttctttgttgttttttttttttcctgtagatacagggtctcgctctgtgacccaggctggtcttaaactcctggcctcaagcgatcctcctgcctctgccttccaaagcactggaattacaagtgtgagccgctgcaacccgccagaaaaaagtgtgcctttcatggccctgtctgggtggctagacacgtgtgtgtgctggtggtcctggcccagccagagtccctgagaggagcatgcatggcctaaggaagtgagcttcagggaacagtgatgaccatcatttcacactcggacccctgcccagatgggtggatggctggcaggagggattccggttttctgcgcctggagaagccctggcaagcggaacctgaaagtatgccctgtccttttcttctcctcagataaaggataggttacagttacgggtgctcttgcgtcgggaagacgtcgtcctggggaggcctatgacccccacccagataggcccaagcctgctgcctatcatgtggcagctctaccctgatggacgataccgatcctcagactccagtttctggcgcttagtgtaccacatcaaggtgagtgtctttctcccagaggtccatcgggtgatcttgggtttgccctcctccatgtctggccttagtggtttatcttccctccatcccagtcccaaaagcattaaaaaatgggggaaatgtacaagtgcagatttctaaaggactttaccagagagaagatagatcctctgaggtctctaacagaaccctacctccacttcctcccagccaccagctagccgcaggaagacatctctggtgggcagtcacaggctgaaggctggtgggaggaggggcagtctccaagagcccctgaaatcctcacacctgggttcctacctgctgtttccagctaggggaaggcgcaggagtgaggaatggagggagtggagggctctggctgatcaatgccttctctctctctctgcctctcagattgacggcgtggaggacatgcttctcgagctgctgccagatgactgatgtatggtgagctccactggagcctgacccctcttagtccacagtggctgtatcagaaagaaagaccaccccttctccatgaaggcagtgctaacccctccccgactgctgccatctgagggtccctagggatgggagaggcttcctggaggcactcatgtctcccttaccacttcgggagccaagggctttgtaggcagccccctttatcgcagctgctcatatctataaagtacttcacaagtttcagctggcactttcatttaccattgcttttttttttttttgagatgagtctggctctgtggcccaggctagagtgtagtggtgcaatctcagctcactgcaagctctgcctcccgggttcacaccattctcctgcctcagccctcggagtagctgggactacaggcgcccgccaccacacctggctaattttttttttttttttttttttttagtagagacggggtttcaccgtgttagccaggatggtctcgatctcctgacctcatgatctgcccgcctcggcctcccaaagtgctgggattacaggcatgagccaccacgtccggccttaccattgctttattaaataagcactggtgcttgattatatcagctgagccagatattagatacgctattgagttttgaggaaataagagtacaaaactcagaaatgagttgaagtatagtgacatcttcagattacagacccaggtgtcagaatttgccttggctcagaaggcctctgggggccatccctgaccactaggcttcccacttagacctgctccagcagcaccacccctcgccactgcctggtcctttccttcacccttgattctgtcttcttttgtccttctccaggtcttggcagcacctgtctcctttcaccccagggcctgagcctggccagcctacaatggggatgttgtgtttctgttcaccttcgtttactatgcctgtgtcttctccaccacgctggggtctgggaggaatggacagacagaggatgagctctacccagggcctgcaggacctgcctgtagcccactctgctcgccttagcactaccactcctgccaaggaggattccatttggcagagcttcttccaggtgcccagctatacctgtgcctcggcttttctcagctggatgatggtcttcagcctctttctgtcccttctgtccctcacagcactagtatttcatgttgcacacccactcagctccgtgaacttgtgagaacacagccgattcacctgagcaggacctctgaaaccctggaccagtggtctcacatggtgctacgcctgcatgtaaacacgcctgcaaacgctgcctgccggtaaacacgcctgcaaacgctgcctgcccgtaaacacgcctgcaaacgctgcctgcccacacaggttcacgtgcagctcaaggaaaggcctgaaaggagcccttatctgtgctcaggactcagaagcctctgggtcagtggtccacatcccgggacgcagcaggaggccaggccggcgagccctgtggatgagccctcagaacccttggcttgcccacgtggaaaagggatagaggttgggtttccccccttttatagatggtcacgcacctgggtgttacaaagttgtatgtggcatgaatactttttgtaatgattgattaaatgcaagatagtttatctaacttcgtgcggaatcagcttctatccttgacttagattctggtggagagaagtgagaataggcagcccccaaataaaaaatattcatggatttatttattttcctttggagataagggactacaattttttccccgtgtaatccaatgcttaaaaccccagtgttcttggaggcatacgttgagaaccattggagtaagcaacctcaaaaatgttgttggcaatgcccggtattccagagccccccctgcctccatctggtcctgccctccttaccctcctcattcccatgtgccctcggccccagccagctctgcctcctccttacctgggcattcaccaggctctgtccttgcacagataacttctatcctctctgctcacaatcccagcagaagcccctcctgcctgaagctgtccctgaccactcaggccccaagcagactcccttccccacctcccatgacctgcagtcacagcagatcccaccctgtgccctgtcctcttgctcccttctcaagaccctgctgcccctcctgtacctctatagacagtagctggacctcagatattaccaggggatgccttcca